(19) United States
(12) Patent Application Publication
(10) Pub. No.: US 2020/0085875 A1
(43) Pub. Date: Mar. 19, 2020

(54) PLATELET VESICLE-ENGINEERED CELLS FOR TARGETED TISSUE REPAIR

(71) Applicant: North Carolina State University, Raleigh, NC (US)

(72) Inventors: Ke Cheng, Raleigh, NC (US); Junnan Tang, Raleigh, NC (US)

(21) Appl. No.: 16/604,680

(22) PCT Filed: Apr. 20, 2018
(86) PCT No.: PCT/US2018/028518

§ 371 Date: Oct. 11, 2019

Related U.S. Application Data
(60) Provisional application No. 62/487,698, filed on Apr. 20, 2017.

(51) Int. Cl. A61K 35/19; A61K 35/28; A61K 35/34; A61K 9/127

(52) U.S. Cl. CPC A61K 35/19; A61K 9/1271; A61K 35/34; A61K 35/28

(57) ABSTRACT

Provided are engineered cells fused with platelet membrane vesicles and capable of specifically targeting a subendothelial site of vascular injury. Also provided are methods of generating a modified cell or extracellular vesicle fused with platelet membrane vesicles or fragments thereof. Further provided are methods for using these engineered cells and vesicles for the treatment of injured tissue accompanied by vascular injury by engraftment with modified cells fused with platelet membrane vesicles. Cells fused with the platelet vesicles can be engineered stem cells such as cardiac or mesenchymal stem cells; the extracellular vesicles fused with the platelet vesicles can be exosomes such as exosomes derived from cardiac or mesenchymal stem cells. The use of the platelet vesicles for targeting sites of vascular injury may also be usefully applied to engineering cells and exosomes that have therapeutic activity for the treatment of vascular injury.

### CROSS REFERENCE TO RELATED APPLICATIONS

This application claims the priority benefit of U.S. Provisional Patent Application Ser. No. 62/487,698 filed on Apr. 20, 2017, which is incorporated herein by reference in its entirety.

### SEQUENCE LISTING

This application contains a sequence listing filed in electronic form as an ASCII.txt file entitled “2214042150_ST25” created on Apr. 11, 2018. The content of the sequence listing is incorporated herein in its entirety.

### TECHNICAL FIELD

The present disclosure is generally related to a modified cell fused with platelet membrane vesicles and capable of specifically targeting a site of vascular injury. The present disclosure is also generally related to methods of generating a modified cell fused with platelet membrane vesicles. The present disclosure further relates to methods of treatment of injured tissue accompanied by vascular injury by engraftment with modified cells fused with platelet membrane vesicles.

### BACKGROUND

The mortality of cardiovascular disease poses an immense burden to the society (Mozaffarian et al., (2015) Circulation 131: 29-322). New therapeutic strategies including stem cells and tissue engineering hold the potential to alter the trajectory of disease progression after an initial insult such as acute myocardial infarction (MI) (Madonna et al., (2016) Eur. Heart J. 37: 1789-1798; Sepantafar et al., (2016) Biotechnol. Adv. 34: 362-379). One big challenge is how to target injected stem cells to an injured zone. Therapeutic benefits are hampered by the low cell retention in the target tissue (Weissman, I. L. (2000) Science 287: 1442-1446). For example, it has been reported that cell retention in the heart is generally less than 10% after several hours, regardless of the cell type and administration route (Cheng et al., (2014) Nat. Commun. 5: 4880; Tongers et al., (2011) Eur. Heart J. 32: 1197-1206). Vascular routes (e.g. intravenous, intracoronary) are relatively safe but have even poorer cell retention rates as compared to direct muscle injection because of the “wash out” effects. This may at least partially explain the inconsistent and marginal therapeutic benefits seen in meta-analysis of stem cell therapy outcomes for heart diseases (van der Spoel. et al., (2011) Cardiovasc. Res. 91: 649-658). Accordingly, novel approaches are urgently needed to better target infused stem cells to the injured heart (Tongers et al., (2011) Eur. Heart J. 32: 1197-1206).

### SUMMARY

Briefly described, one aspect of the disclosure encompasses embodiments of a composition comprising: (a) a platelet membrane-derived vesicle or a fragment thereof; and (b) an animal or human cell, a plurality of said cells, or an extracellular vesicle derived from the cell, wherein the platelet-derived membrane vesicle or fragments thereof can be fused into the outer membrane of the cell or plurality of said cells or encapsulates the extracellular vesicle, and wherein the composition can be characterized as having specific binding affinity for at least one component of a vascular subendothelial matrix or vascular cell.

In some embodiments of this aspect of the disclosure, the extracellular vesicle can be an exosome.

In some embodiments of this aspect of the disclosure, the animal or human cell can be a stem cell.

In some embodiments of this aspect of the disclosure, the stem cell can be a cardiac stem cell or a mesenchymal stem cell.

In some embodiments of this aspect of the disclosure, the animal or human cell can have an outer membrane engineered to have platelet-derived polypeptide cell-surface markers.

In some embodiments of this aspect of the disclosure, the animal or human cell can be isolated from an animal or human tissue, a cultured cell, or a cryopreserved cell.

In some embodiments of this aspect of the disclosure, the stem cell can be from a cultured cardiosphere from a cardiac tissue.

In some embodiments of this aspect of the disclosure, the extracellular vesicle can be derived from a cardiac stem cell or a mesenchymal stem cell.

In some embodiments of this aspect of the disclosure, the animal or human cell, plurality of cells, or the extracellular vesicle can be derived from the same animal or human subject as the platelet-derived membrane vesicle.

In some embodiments of this aspect of the disclosure, the animal or human cell, plurality of cells, or the extracellular vesicle can be derived from a different animal or human subject as the platelet-derived membrane vesicle.

In some embodiments of this aspect of the disclosure, (i) the animal or human cell, plurality of cells, or the extracellular vesicle and (ii) the platelet-derived membrane vesicle of the composition can both be derived from the animal or human subject that is a recipient of the composition for treatment of a vascular injury.

In some embodiments of this aspect of the disclosure, at least one of (a) the animal or human cell, plurality of cells, or the extracellular vesicle and (b) the platelet-derived membrane vesicle or fragments thereof of the composition can be derived from the animal or human subject that is a recipient of the composition for treatment of a vascular injury.

In some embodiments of this aspect of the disclosure, the composition can be admixed with a pharmaceutically acceptable carrier.

Another aspect of the disclosure encompasses embodiments of a method of generating a population of engineered animal or human cells or extracellular vesicles derived from said cells, the method comprising the step of mixing a population of platelet-derived membrane vesicles or fragments thereof and a population of cells or extracellular vesicles and thereby fusing the platelet-derived membrane vesicles with the outer membranes of the cells or encapsulating the extracellular vesicles, wherein said cells or extracellular vesicles can be isolated from an animal or human tissue or biofluid, cultured cells, or cryopreserved cells.

In some embodiments of this aspect of the disclosure, the method can further comprise the steps of: (i) obtaining a suspension of platelets isolated from the plasma of an animal or human subject; and (ii) sonicating the suspension of platelets to generate a population of platelet-derived membrane vesicles or fragments thereof.

In some embodiments of this aspect of the disclosure, the method can further comprise the step of incubating the cells, or extracellular vesicles derived therefrom, together with the platelet-derived membrane vesicles in the presence of polyethylene glycol (PEG) or extruding the cells, or extracellular vesicles derived therefrom, together with the platelet-derived membrane vesicles or fragments thereof, and thereby fusing the platelet-derived membrane vesicles or fragments thereof with the outer membranes of the cells or encapsulating the extracellular vesicles.

In some embodiments of this aspect of the disclosure, the extracellular vesicle can be an exosome.

In some embodiments of this aspect of the disclosure, the animal or human cells can be stem cells.

In some embodiments of this aspect of the disclosure, the stem cells can be derived from a cardiac tissue.

In some embodiments of this aspect of the disclosure, the method can further comprise the step of obtaining the stem cells from a cultured tissue explant derived from a cardiac tissue.

In some embodiments of this aspect of the disclosure, the method can further comprise the step of obtaining the platelet-derived membrane vesicles or fragments thereof and the cells, or the extracellular vesicles derived from said cells, from the same animal or human subject.

In some embodiments of this aspect of the disclosure, the method can further comprise the step of obtaining the platelet-derived membrane vesicles or fragments thereof and the cells, or the extracellular vesicles derived from said cells, from different individual animal or human subjects.

Yet another aspect of the disclosure encompasses embodiments of a method of repairing a tissue injury in an animal or human subject, the method comprising administering to a recipient animal or human patient having a tissue injury a composition comprising a population of engineered cells or extracellular vesicles derived from said cells, wherein the engineered cells or extracellular vesicles comprise a platelet-derived membrane vesicle or fragments thereof fused into the outer membrane of the cell or encapsulating the extracellular vesicle or vesicles, and wherein the engineered cells or extracellular vesicles selectively target the subendothelial matrix or a vascular cell at the site of the tissue injury when administered to a recipient animal or human.

In some embodiments of this aspect of the disclosure, the engineered cells can be engineered stem cells or extracellular vesicles derived from said stem cells and the tissue injury of the subject can be to a tissue of the cardiovascular system.

In some embodiments of this aspect of the disclosure, the engineered cells can be cardiac or mesenchymal stem cells and the extracellular vesicles can be derived from said cardiac or mesenchymal stem.

In some embodiments of this aspect of the disclosure, the extracellular vesicles can be exosomes.

In some embodiments of this aspect of the disclosure, the tissue injury can be accompanied by an injury to a blood vessel.

In some embodiments of this aspect of the disclosure, the tissue injury can be of neural tissue, muscular tissue, cardiac tissue, or hepatic tissue, and wherein the engineered cells migrate to the injured tissue.

In some embodiments of this aspect of the disclosure, the engineered cells or engineered extracellular vesicles can be derived from the same animal or human subject as the platelet-derived membrane vesicles or fragments thereof.

In some embodiments of this aspect of the disclosure, the engineered cells or engineered extracellular vesicles are not derived from the same animal or human subject as the platelet-derived membrane vesicles or fragments thereof.

In some embodiments of this aspect of the disclosure, at least one of (i) the engineered cells or engineered extracellular vesicles derived therefrom and (ii) the platelet-derived membrane vesicles or fragments thereof, are derived from the recipient animal or human patient.

### BRIEF DESCRIPTION OF THE DRAWINGS

Further aspects of the present disclosure will be more readily appreciated upon review of the detailed description of its various embodiments, described below, when taken in conjunction with the accompanying drawings.

FIGS. 1A-1G illustrate platelet binding to myocardial infarction (MI) and the derivation of platelet-derived membrane nanovesicles.

FIG. 1A is a scheme showing the animal study design to test the natural MI-binding ability of platelets.

FIG. 1B is a digital image showing representative ex vivo fluorescent imaging showing binding of intravenously injected CM-DiI labeled platelets in hearts with or without ischemia/reperfusion (I/R) injury.

FIG. 1C is a digital image showing representative fluorescent images showing the targeting of CM-DiI labeled platelets (red) to the MI area. Scale bar=100 μm.

FIGS. 1D and 1E are digital images showing collected rat red blood cells (FIG. 1D) under light microscope, which demonstrates a distinctive morphology from platelets (FIG. 1E). Scale bar=10 μm.

FIG. 1F is a digital image showing a transmission electron micrograph of platelet nanovesicles. Scale bar=100 nm.

FIG. 1G is a graph showing the size distribution of platelets membrane vesicles by NanoSight examination.

FIGS. 2A-2F illustrate the generation and characterization of platelet nanovesicle-decorated cardiac stem cells (PNV-CSCs).

FIG. 2A is a scheme showing the overall study design.

FIGS. 2B and 2C are digital images showing Red fluorescent CM-DiI-labeled CSCs (FIG. 2B) were fused with the green fluorescent DiO-labeled platelet nanovesicles to form PNV-CSCs (FIG. 2C). Scale bar=20 μm.

FIG. 2D is a digital image showing the co-culture of CSCs and PNV-CSCs. Scale bar=20 μm.

FIG. 2E is a digital image showing a Western blotting analysis revealing the expressions of platelet-specific markers including CD42b (GPIbα), GPVI and CD36 (GPIV) in platelets, platelet-vesicles, PNV-CSCs but not in CSCs.

FIG. 2F is a digital image showing immunocytochemistry (ICC) staining confirmed CD42b (GPIbα) and GPVI expressions in PNV-CSCs (upper panel), but not in CSCs (lower panel). Scale bar=200 μm

FIG. 2G is a digital image illustrating a flow cytometry analysis of surface markers expressed on CSCs and PNV-CSCs.

FIG. 2H is a graph illustrating the quantitative analysis of surface marker expressions on CSCs and PNV-CSCs.

FIGS. 3A-3E illustrate the effects of platelet nanovesicles (PNV) decoration on cardiac stem cells (CSCs) viability and functions. FIG. 3A is a digital image showing representative fluorescent micrographs showing live (Calcien-AM: green) and dead (EthD: red) staining of PNV-CSCs or CSCs cultured on tissue culture plate (TCP) for 7 days. Scale bar=200 μm.

FIG. 3B is a graph showing pooled data of cell viability (n=3 per group).

FIG. 3C is a graph showing CCK8 assay measurement of proliferation of PNV-CSCs or CSCs cultured on TCP (n=3 per group at each time point).

FIG. 3D is a graph showing trans-well migration assay showing the migration potencies of PNV-CSCs or CSCs. (n=3 per group at each time point).

FIG. 3E is a graph showing the ELISA determination of the release of growth factors including IGF-1, SDF-1, VEGF, and HGF in the conditioned media (CM) from CSCs or PNV-CSCs (n=3 per group). Values are mean±s.d. Two-tailed t test for comparison.

FIGS. 4A-4J illustrate augmented binding of platelet nanovesicle-decorated cardiac stem cells (PNV-CSCs) to damaged rodent vasculatures.

FIG. 4A illustrates a schematic showing experimental design for rat aorta binding.

FIGS. 4B-4E illustrate a series of digital images showing representative fluorescent micrographs showing the adherence of DiI-labeled PNV-CSCs and CSCs on control aortas (FIGS. 4B and 4C) or denuded aortas (FIGS. 4D and 4E). Scale bar=1 mm.

FIG. 4F is a schematic showing PNV-CSCs or CSCs seeded on collagen-coated tissue culture slides.

FIGS. 4G and 4H illustrate a pair of digital images showing representative fluorescent images of the binding of DiI-labeled PNV-CSCs (FIG. 4G) or control CSCs (FIG. 4H) on collagen-coated tissue culture slides. Scale bar=50 μm.

FIGS. 4I and 4J are graphs illustrating quantitative analysis of cell binding (n=3 experiments per group). * indicated P<0.05 when compared to “CSC” group. All values are mean±s.d. Two-tailed t test for comparison between the two groups.

FIGS. 5A-5F illustrate that platelet nanovesicle (PNV) decoration boosts cardiac stem cells (CSCs) retention in the myocardial infarction (MI) heart.

FIG. 5A is a schematic showing the animal study design.

FIG. 5B is a digital image showing representative ex vivo fluorescent imaging of ischemia/reperfusion (I/R) rat hearts 24 h after intracoronary infusion of DiI-labeled PNV-CSCs or CSCs.

FIG. 5C is a graph showing that qPCR analysis revealed higher retention rates of PNV-CSCs (red bar) as compared to those of CSCs (blue bar) (n=3 animals/hearts per group).

FIGS. 5D and 5E are a pair of digital images of representative fluorescent micrographs showing engrafted CSCs (FIG. 5D) or PNV-CSCs (FIG. 5E) in the post-MI hearts. Scale bar=50 μm.

FIG. 5F is a graph showing quantitative analysis of cell engraftment by histology (n=3 animals [3 sections for each animal] per group). * indicated P<0.05 when compared to “CSC” group. All values are mean±s.d. Two-tailed t test for comparison.

FIGS. 6A-6I illustrate that platelet nanovesicle (PNV) decoration augments the therapeutic benefits of cardiac stem cells (CSCs). Scale bar=2 mm.

FIG. 6A illustrates a series of digital images showing representative Masson's trichrome-stained myocardial sections 4 weeks after treatment.

FIGS. 6B and 6C are a pair of graphs illustrating quantitative analyses of viable myocardium and scar size from the Masson's trichrome images (n=5 animals per group).

FIGS. 6D and 6E are a pair of graphs illustrating left ventricular ejection fractions (LVEFs) measured by echocardiography at baseline (4 h post-MI) and 4 weeks afterward (n=6 animals per group). * indicated P<0.05 when compared to “Control” group; #** indicated P<0.05 when compared to “CSC” group. All values are mean±s.d. One-way ANOVA with post-hoc Bonferroni test.

FIGS. 6F and 6G are a pair of graphs illustrating left ventricular end diastolic volume (LVEDVs) measured by echocardiography at baseline (4 h post-MI) and 4 weeks afterward (n=6 animals per group). * indicated P<0.05 when compared to “Control” group; # indicated P<0.05 when compared to “CSC” group. All values are mean±s.d. One-way ANOVA with post-hoc Bonferroni test.

FIGS. 6H and 6I are a pair of graphs illustrating left ventricular end systolic volume (LVESVs) measured by echocardiography at baseline (4 h post-MI) and 4 weeks afterward (n=6 animals per group). * indicated P<0.05 when compared to “Control” group; # indicated P<0.05 when compared to “CSC” group. All values are mean±s.d. One-way ANOVA with post-hoc Bonferroni test.

FIGS. 7A-7C illustrate that PNV-CSC therapy promotes myocyte proliferation and angiogenesis.

FIG. 7A shows representative images showing Ki67-positive cardiomyocyte nuclei (red, with red arrows) in control PBS-, CSC, or PNV-CSC-treated hearts at 4 weeks. The numbers of Ki67-positive nuclei were quantified. n=3-4 animals per group. Scale Bar, 20 μm.

FIG. 7B shows representative images showing lectin-labeled blood vessels (green) in control PBS- and CSC-, or PNV-CSC-treated hearts at 4 weeks. The lectin fluorescent intensities were quantified. n=3-4 animals per group. Scale Bar, 100 μm.

FIG. 7C shows representative images showing arterioles stained with alpha smooth muscle actin (αSMA, red) in PBS- and CSC-, or PNV-CSC-treated hearts at 4 weeks. The numbers of aSMA positive vasculatures were quantified. n=3-4 animals per group. Scale Bar, 50 μm. * indicates P<0.05 when compared to Control group. #** indicates P<0.05 when compared to Control or CSC group. All data are mean±s.d. Comparisons were performed using one-way ANOVA followed by post-hoc Bonferroni test.

FIGS. 8A-8E illustrate the role of CD42b in targeting PNV-CSCs to MI injury.

FIG. 8A shows representative fluorescent micrographs showing the adherence of anti-CD42b or isotype antibody pre-treated PNV-CSCs on denuded rat aortas.

FIG. 8B is a graph illustrating quantitation of binding (n=3 samples per group).

FIG. 8C is a digital image showing representative ex vivo fluorescent imaging of ischemia/reperfusion (I/R) rat hearts 24 h after intracoronary infusion of anti-CD42b or isotype antibody pre-treated PNV-CSCs.

FIG. 8D is a graph illustrating quantitation of cell retention by qPCR (n=3 animals per group.

FIG. 8E illustrates representative Masson's trichrome-stained myocardial sections 4 weeks after treatment. Quantitative analyses of viable myocardium and scar size from the Masson's trichrome images (n=5 animals per group). Left ventricular ejection fractions (LVEFs) measured by echocardiography at baseline (4 h post-MI) and 4 weeks afterward (n=6 animals per group). * indicated P<0.05 when compared to “PNV-CSC+iso Ab” group. All values are mean±s.d. Comparison between two groups is performed with two-tailed t tests.

FIGS. 9A-9H illustrate the targeting effects of PNV-CSCs in a pig model of myocardium infarction (MI) injury.

FIG. 9A is a scheme showing the overall design of the pig study.

FIG. 9B is a series of digital x-ray images illustrating the creation of the pig MI model.

FIG. 9C is a pair of ECG images showing the change in ECG after MI creation.

FIG. 9D is a photo showing an excised pig heart and a scheme for further histological processing.

FIG. 9E is a pair of fluorescent images showing the retention of CSCs and PNV-CSCs in the pig heart after intracoronary injections.

FIG. 9F is a graph showing the quantitation of fluorescent signals measured from the experiments and images in FIG. 9E. # indicated P<0.05 when compared to “CSC” group. All values are mean±s.d. Comparison between two groups is performed with two-tailed t tests.

FIG. 9G is a pair of fluorescent images showing tetrazolium chloride staining of heart sections after injection with CSCs or PNV-CSCs.

FIG. 9H is a graph showing the quantitation of infarct size measured from the experiments and images in FIG. 9G. N.S. indicated P>0.05 when compared to “CSC” group. All values are mean±s.d. Comparison between two groups is performed with two-tailed t tests.

FIG. 10 is a scheme showing the production of platelet vesicle engineered stem cell exosomes (EXOs). Exosomes can be integrated into platelet vesicles through methods such as extrusion, vesicle fusion, and sonication known in the art.

FIG. 11 is a series of digital electron photomicrograph images of a platelet membrane vesicle (PV), exosomes (EXO), and platelet membrane vesicle coated stem cell exosomes.

FIG. 12 is a series of traces showing the size distribution of platelet membrane vesicle (PV), exosomes (EXO), and platelet membrane vesicle coated stem cell exosomes.

FIG. 13 is a series of digital high resolution fluorescent microscopic images of a platelet membrane vesicle (PV), exosome (EXO), and platelet membrane vesicle coated stem cell exosomes.

FIG. 14 is a schematic of the targeting effects of PV-EXOs in a human coronary vessel injury model. Human coronary vessels were isolated from a failing heart donor. The vessels were denuded from the luminal side by a pair of surgical forceps. PV-EXOs or control EXOs were run through the vessels and binding efficiency was checked by fluorescent microscopy.

FIG. 15 is a graph illustrating that PV-EXOs have enhanced binding to injured blood vessels when compared to control EXOs.

FIG. 16 illustrates the targeting effects of PV-EXOs in a porcine model of myocardial infarction. Ischemia-reperfusion injury was created in pigs. PV-EXOs or control EXOs were injected and the retention of those exosomes in the pig hearts were examined by ex vivo fluorescent heart imaging. PV-EXOs had enhanced cardiac retention than control EXOs (right-hand graph).

FIG. 17 is a series of digital images lustrating the therapeutic effects of PV-EXOs in a rat model of myocardial infarction. Ischemia-reperfusion injury was created in rats. PV-EXOs or control EXOs were injected and therapeutic benefits were gauged by Masson's trichrome staining of heart sections.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of medicine, organic chemistry, biochemistry, molecular biology, pharmacology, and the like, which are within the skill of the art. Such techniques are explained fully in the literature.

It must be noted that, as used in the specification and the appended claims, the singular forms “a,” “an,” and “the” include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent.

As used herein, the following terms have the meanings ascribed to them unless specified otherwise. In this disclosure, “comprises,” “comprising,” “containing” and “having” and the like can have the meaning ascribed to them in U.S. patent law and can mean “includes,” “including,” and the like; “consisting essentially of” or “consists essentially” or the like, when applied to methods and compositions encompassed by the present disclosure refers to compositions like those disclosed herein, but which may contain additional structural groups, composition components or method steps (or analogs or derivatives thereof as discussed above). Such additional structural groups, composition components or method steps, etc., however, do not materially affect the basic and novel characteristic(s) of the compositions or methods, compared to those of the corresponding compositions or methods disclosed herein.

Prior to describing the various embodiments, the following definitions are provided and should be used unless otherwise indicated.

Numerical ranges recited herein by endpoints include all numbers and fractions subsumed within that range (e.g., 1 to 5 includes 1, 1.5, 2, 2.75, 3, 3.90, 4, and 5). It is also to be understood that all numbers and fractions thereof are presumed to be modified by the term “about.” “About” as used herein indicates that a number, amount, time, etc., is not exact or certain but reasonably close to or almost the same as the stated value. Therefore, the term “about” means plus or minus 0.1 to 50%, 5-50%, or 10-40%, preferably 10-20%, more preferably 10% or 15%, of the number to which reference is being made. Further, it is to be understood that “a”, “an,” and “the” include plural referents unless the content clearly dictates otherwise. Thus, for example, reference to a composition comprising “a compound” includes a mixture of two or more compounds.
Abbreviations
Myocardial infarction, MI; Platelet nanovesicle, PNV; cardiac stem cell, CSC; cardiosphere-derived cells, CDCs
Definitions

The term “cell” as used herein refers to an animal or human cell. The engineered cells of the disclosure can be removed (isolated) from a tissue and mixed with platelet-derived membrane vesicles without culturing of the cells, or after removal from a tissue or fluid of an animal or human may be cultured to increase the population size of the cells. If not immediately required for incorporation of the platelet-derived membrane vesicles, the animal or human cells may be maintained in a viable state either by culturing by serial passage in a culture medium or cryopreserved by methods well-known in the art. The cells may be obtained from the animal or human individual that has received an injury desired to be repaired by administration of the engineered cells of the disclosure or from a different individual.

Cells, and their extracellular vesicle such as an exosomes that are contemplated for use in the methods of the present disclosure may be derived from the same subject to be treated (autologous to the subject) or they may be derived from a different subject, preferably of the same species, (allogeneic to the subject).

Commercially available media may be used for the growth, culture and maintenance of mesenchymal stem cells. Such media include but are not limited to Dulbecco's modified Eagle's medium (DMEM). Components in such media that are useful for the growth, culture and maintenance of mesenchymal stem cells include but are not limited to amino acids, vitamins, a carbon source (natural and non-natural), salts, sugars, plant derived hydrolysates, sodium pyruvate, surfactants, ammonia, lipids, hormones or growth factors, buffers, non-natural amino acids, sugar precursors, indicators, nucleosides and/or nucleotides, butyrate or organics, DMSO, animal derived products, gene inducers, non-natural sugars, regulators of intracellular pH, betaine or osmoprotectant, trace elements, minerals, non-natural vitamins. Additional components that can be used to supplement a commercially available tissue culture medium include, for example, animal serum (e.g., fetal bovine serum (FBS), fetal calf serum (FCS), horse serum (HS)), antibiotics (e.g., including but not limited to, penicillin, streptomycin, neomycin sulfate, amphotericin B, blasticidin, chloramphenicol, amoxicillin, bacitracin, bleomycin, cephalosporin, chlortetracycline, zeocin, and puromycin), and glutamine (e.g., L-glutamine). Mesenchymal stem cell survival and growth also depends on the maintenance of an appropriate aerobic environment, pH, and temperature. Mesenchymal stem cells can be maintained using methods known in the art (see for example Pittenger et al., (1999) Science 284:143-147).

The term “extracellular vesicle” as used herein can refers to a membrane vesicle secreted by cells that may have a larger diameter than that referred to as an “exosome”. Extracellular vesicles (alternatively named “microvesicle” or “membrane vesicle”) may have a diameter (or largest dimension where the particle is not spheroid) of between about 10 nm to about 5000 nm (e.g., between about 50 nm and 1500 nm, between about 75 nm and 1500 nm, between about 75 nm and 1250 nm, between about 50 nm and 1250 nm, between about 30 nm and 1000 nm, between about 50 nm and 1000 nm, between about 100 nm and 1000 nm, between about 50 nm and 750 nm, etc.). Typically, at least part of the membrane of the extracellular vesicle is directly obtained from a cell (also known as a donor cell). Extracellular vesicles suitable for use in the compositions and methods of the present disclosure may originate from cells by membrane inversion, exocytosis, shedding, blebbing, and/or budding. Extracellular vesicles may originate from the same population of donor cells yet different subpopulations of extracellular vesicles may exhibit different surface/lipid characteristics. Alternative names for extracellular vesicles include, but are not limited to, exosomes, ectosomes, membrane particles, exosome-like particles, and apoptotic vesicles. Depending on the manner of generation (e.g., membrane inversion, exocytosis, shedding, or budding), the extracellular vesicles contemplated herein may exhibit different surface/lipid characteristics.

The term “exosomes” as used herein refers to small secreted vesicles (typically about 30 nm to about 150 nm(or largest dimension where the particle is not spheroid)) that may contain, or have present in their membrane, nucleic acid, protein, or other biomolecules and may serve as carriers of this cargo between diverse locations in a body or biological system. The term “exosomes” as used herein advantageously refers to extracellular vesicles that can have therapeutic properties, including, but not limited to stem cell exosomes such as cardiac stem cell or mesenchymal stem cells.

Exosomes may be isolated from a variety of biological sources including mammals such as mice, rats, guinea pigs, rabbits, dogs, cats, bovine, horses, goats, sheep, primates or humans. Exosomes can be isolated from biological fluids such as serum, plasma, whole blood, urine, saliva, breast milk, tears, sweat, joint fluid, cerebrospinal fluid, semen, vaginal fluid, ascetic fluid and amniotic fluid. Exosomes may also be isolated from experimental samples such as media taken from cultured cells (“conditioned media”, cell media, and cell culture media).

Exosomes may also be isolated from tissue samples such as surgical samples, biopsy samples, and cultured cells. When isolating exosomes from tissue sources it may be necessary to homogenize the tissue in order to obtain a single cell suspension followed by lysis of the cells to release the exosomes. When isolating exosomes from tissue samples it is important to select homogenization and lysis procedures that do not result in disruption of the exosomes.

Exosomes may be isolated from freshly collected samples or from samples that have been stored frozen or refrigerated. Although not necessary, higher purity exosomes may be obtained if fluid samples are clarified before precipitation with a volume-excluding polymer, to remove any debris from the sample. Methods of clarification include centrifugation, ultracentrifugation, filtration or ultrafiltration.

The genetic information within the extracellular vesicle such as an exosome may easily be transmitted by fusing to the membranes of recipient cells, and releasing the genetic information into the cell intracellularly. Though exosomes as a general class of compounds represent great therapeutic potential, the general population of exosomes are a combination of several class of nucleic acids and proteins which have a constellation of biologic effects both advantageous and deleterious. In fact, there are over 1000 different types of exosomes.

The term “stem cell” as used herein refers to cells that have the capacity to self-renew and to generate differentiated progeny. The term “pluripotent stem cells” refers to stem cells that have complete differentiation versatility, i.e., the capacity to grow into any of the fetal or adult mammalian body's approximately 260 cell types. For example, pluripotent stem cells have the potential to differentiate into three germ layers: endoderm (e.g., blood vessels), mesoderm (e.g., muscle, bone and blood) and ectoderm (e.g., epidermal tissues and nervous system), and therefore, can give rise to any fetal or adult cell type. The term “cardiac stem cells” refers to stem cells obtained from or derived from cardiac tissue. The term “cardiosphere-derived cells (CDCs)” as used herein refers to undifferentiated cells that grow as self-adherent clusters from subcultures of postnatal cardiac surgical biopsy specimens. CDCs can express stem cell as well as endothelial progenitor cell markers, and typically possess properties of adult cardiac stem cells. For example, human CDCs can be distinguished from human cardiac stem cells in that human CDCs typically do not express multidrug resistance protein 1 (MDR1; also known as ABCB1), CD45 and CD133 (also known as PROM1). CDCs are capable of long-term self-renewal, and can differentiate in vitro to yield cardiomyocytes or vascular cells after ectopic (dorsal subcutaneous connective tissue) or orthotopic (myocardial infarction) transplantation in SCID beige mouse.

The terms “cardiac progenitor cells” and “cardiac stem cells” as used herein can refer to a population of progenitor cells derived from human heart tissue. In some aspects the present disclosure, of the cardiac progenitor (stem) cells, at least 3%, 5%, 7%, 10%, 12%, or 15%, e.g., 3-50%, 3-20%, 3-10%, 5-30%, 5-10%, etc., of the cells express |sl1. In some aspects, CPCs comprise about 10%, 15%, 20%, 30%, 40%, or 50%, e.g., 10-50%, 10-40%, 10-30%, 15-40%, etc., GATA4 expressing cells. In some aspects, cardiac stem cells comprise about 5%, 8%, 10%, 13%, or 15%, e.g., 8-15%, 5-15%, 5-13%, etc., NKX2.5 expressing cells. Before fusion with platelet membrane vesicles by the methods of the present disclosure, cardiac stem cells are unmodified cells in that recombinant nucleic acids or proteins have not been introduced into them or the Sca-1⁺, CD45-cell from which it is derived. As such, cardiac stem cells as isolated from cardiac tissue are non-transgenic, or in other words have not been genetically modified. For example, expression of genes such as lsl1, GATA4, and NKX2.5 in CPCs is from the endogenous gene. Cardiac stem cells may comprise Sca-1+, CD45-cells, c-kit⁺ cells, CD90⁺ cells, CD133⁺ cells, CD31⁺ cells, Flk1⁺ cells, GATA4⁺ cells, or NKX2.5+ cells, or combinations thereof. In some aspects, cardiac stem cells comprise about 50% GATA4 expressing cells. In some aspects, cardiac stem cells comprise about 15% NKX2.5 expressing cells. Cardiac stem cells can replicate and are capable of differentiating into endothelial cells, cardiomyocytes, smooth muscle cells, and the like.

The term “cardiac cells” as used herein refers to any cells present in the heart that provide a cardiac function, such as heart contraction or blood supply, or otherwise serve to maintain the structure of the heart. Cardiac cells as used herein encompass cells that exist in the epicardium, myocardium or endocardium of the heart. Cardiac cells also include, for example, cardiac muscle cells or cardiomyocytes; cells of the cardiac vasculatures, such as cells of a coronary artery or vein. Other non-limiting examples of cardiac cells include epithelial cells, endothelial cells, fibroblasts, cardiac conducting cells and cardiac pacemaking cells that constitute the cardiac muscle, blood vessels and cardiac cell supporting structure.

The term “cardiac function” as used herein refers to the function of the heart, including global and regional functions of the heart. The term “global” cardiac function as used herein refers to function of the heart as a whole. Such function can be measured by, for example, stroke volume, ejection fraction, cardiac output, cardiac contractility, etc. The term “regional cardiac function” refers to the function of a portion or region of the heart. Such regional function can be measured, for example, by wall thickening, wall motion, myocardial mass, segmental shortening, ventricular remodeling, new muscle formation, the percentage of cardiac cell proliferation and programmed cell death, angiogenesis and the size of fibrous and infarct tissue. Techniques for assessing global and regional cardiac function are known in the art. For example, techniques that can be used to measure regional and global cardiac function include, but are not limited to, echocardiography (e.g., transthoracic echocardiogram, transesophageal echocardiogram or 3D echocardiography), cardiac angiography and hemodynamics, radionuclide imaging, magnetic resonance imaging (MRI), sonomicrometry and histological techniques.

The term “cardiac tissue” as used herein refers to tissue of the heart, for example, the epicardium, myocardium or endocardium, or portion thereof, of the heart. The term “injured” cardiac tissue as used herein refers to a cardiac tissue that is, for example, ischemic, infarcted, reperfused, or otherwise focally or diffusely injured or diseased. Injuries associated with a cardiac tissue include any areas of abnormal tissue in the heart, including any areas caused by a disease, disorder or injury and includes damage to the epicardium, endocardium and/or myocardium. Non-limiting examples of causes of cardiac tissue injuries include acute or chronic stress (e.g., systemic hypertension, pulmonary hypertension or valve dysfunction), atheromatous disorders of blood vessels (e.g., coronary artery disease), ischemia, infarction, inflammatory disease and cardiomyopathies or myocarditis.

The term “mesenchymal stem cells (MSCs)” as used herein refers to progenitor cells having the capacity to differentiate into neuronal cells, adipocytes, chondrocytes, osteoblasts, myocytes, cardiac tissue, and other endothelial and epithelial cells. (See for example Wang, (2004) Stem Cells 22: 1330-1337; McElreavey (1991) Biochem. Soc. Trans. 1: 29s; Takechi (1993) Placenta 14: 235-245; Yen (2005) Stem Cells; 23: 3-9). These cells have been characterized to express, and thus be positive for, one or more of CD13, CD29, CD44, CD49a, b, c, e, f, CD51, CD54, CD58, CD71, CD73, CD90, CD102, CD105, CD106, CDw119, CD120a, CD120b, CD123, CD124, CD126, CD127, CD140a, CD166, P75, TGF-131R, TGF-1311R, HLA-A, B, C, SSEA-3, SSEA-4, D7 and PD-L1.

Mesenchymal stem cells may be harvested from a number of sources including, but not limited to, bone marrow, blood, periosteum, dermis, umbilical cord blood and/or matrix (e.g., Wharton's Jelly), and placenta. Example of methods for obtaining mesenchymal stem cells reference can be found in U.S. Pat. No. 5,486,359.

The term “engraftment” as used herein refers to the process by which transplanted stem cells (e.g., autologous stem cells) are accepted by a host tissue, survive and persist in that environment. In certain embodiments, the transplanted stem cells further reproduce.

The terms “generate”, “generation”, and “generating” as used herein shall be given their ordinary meaning and shall refer to the production of new cells in a subject and optionally the further differentiation into mature, functioning cells. Generation of cells may comprise regeneration of the cells. Generation of cells comprises improving survival, engraftment and/or proliferation of the cells.

The terms “regenerate,” “regeneration” and “regenerating” as used herein refer to the process of growing and/or developing new cardiac tissue in a heart or cardiac tissue that has been injured, for example, injured due to ischemia, infarction, reperfusion, or other disease. Tissue regeneration may comprise activation and/or enhancement of cell proliferation. Cardiac tissue regeneration comprises activation and/or enhancement of cell migration.

The term “cell therapy” as used herein refers to the introduction of new cells into a tissue in order to treat a disease and represents a method for repairing or replacing diseased tissue with healthy tissue.

The term “derived from” as used herein refers to cells or a biological sample (e.g., blood, tissue, bodily fluids, etc.) and indicates that the cells or the biological sample were obtained from the stated source at some point in time. For example, a cell derived from an individual can represent a primary cell obtained directly from the individual (i.e., unmodified). In some instances, a cell derived from a given source undergoes one or more rounds of cell division and/or cell differentiation such that the original cell no longer exists, but the continuing cell (e.g., daughter cells from all generations) will be understood to be derived from the same source. The term includes directly obtained from, isolated and cultured, or obtained, frozen, and thawed. The term “derived from” may also refer to a component or fragment of a cell obtained from a tissue or cell.

The term “isolating” or “isolated” when referring to a cell or a molecule (e.g., nucleic acids or protein) indicates that the cell or molecule is or has been separated from its natural, original or previous environment. For example, an isolated cell can be removed from a tissue derived from its host individual, but can exist in the presence of other cells (e.g., in culture), or be reintroduced into its host individual.

The term “culturing” as used herein refers to growing cells or tissue under controlled conditions suitable for survival, generally outside the body (e.g., ex vivo or in vitro). The term includes “expanding,” “passaging,” “maintaining,” etc. when referring to cell culture of the process of culturing. Culturing cells can result in cell growth, differentiation, and/or division.

The term “disaggregating” includes separating, dislodging, or dissociating cells or tissue using mechanical or enzymatic disruption to isolate single cells or small clusters of cells. In some instances, enzymatic disruption can be replaced with one of more enzyme alternatives having substantially the same effect as the enzyme.

The term “clonal” refers to a cell or a group of cells that have arisen from a single cell through numerous cycles of cell division. The cells of a clonal population are genetically identical. A clonal population can be a heterogeneous population such that the cells can express a different set of genes at a specific point in time.

The term “progenitor cell” as used herein refers to a cell that has the capacity to differentiate into a specific type of cell, as well as replicate to generate a daughter cell substantially equivalent to itself. In some instances, a progenitor cell undergoes limited self-renewal such that it does not self-replicate indefinitely.

The term “self-renewal” or “self-renewing” as used herein refers to the ability of a cell to divide through numerous cycles of cell division and generate a daughter with the same characteristics as the parent cell. The other daughter cell can have characteristics different from its parent cell. The term includes the ability of a cell to generate an identical genetic copy of itself (e.g., clone) by cell division. For example, a self-renewing cardiac progenitor cell can divide to form one daughter cardiac progenitor cell and another daughter cell committed to differentiation to a cardiac lineage such as an endothelial, smooth muscle or cardiomyocyte cell. In some instances, a self-renewing cell does not undergo cell division forever.

The term “multipotent” refers to a cell having the potential to differentiate into multiple, yet a limited number of cell types or cell lineages. Typically, these cells are considered unspecialized cells that have the ability to self-renew and become specialized cells with specific functions and characteristics.

The term “tissue injury” as used herein refers to damage to a vascularized tissue of an animal or human, wherein the damage is adjacent to, or in close proximity to, a blood vessel that has also undergone injury, and in particular loss of endothelial cells lining the lumen of the blood vessel. For example, but not intended to be limiting, vascular ischemia can result in both loss of vascular endothelial cells to expose the underlying subendothelial matrix. The loss of adequate blood flow can result in loss of cell viability in such as cardiac tissue, brain or neurological tissue that is in contact with the occluded blood vessel.

The term “endothelial cell” refers to a cell necessary for the formation and development of new blood vessel from pre-existing vessels (e.g., angiogenesis). Typically, endothelial cells are the thin layer of cells that line the interior surface of blood vessels and lymphatic vessels. Endothelial cells are involved in various aspects of vascular biology, including atherosclerosis, blood clotting, inflammation, angiogenesis, and control of blood pressure.

The term “smooth muscle cell” refers to a cell comprising non-striated muscle (e.g., smooth muscle). Smooth muscle is present within the walls of blood vessels, lymphatic vessels, cardiac muscle, urinary bladder, uterus, reproductive tracts, gastrointestinal tract, respiratory tract, and iris of the eye.

The term “cardiomyocyte cell” refers to a cell comprising striated muscle of the walls of the heart. Cardiomyocytes can contain one or more nuclei.

The term “cardiosphere” refers to a cluster of cells derived from heart tissue or heart cells. In some instances, a cardiosphere includes cells that express stem cell markers (e.g., c-Kit, Sca-1, and the like) and differentiating cells expressing myocyte proteins and the gap protein (connexin 43).

The term “autologous” refers to deriving from or originating in the same subject or patient. An “autologous transplant” refers to collection (e.g., isolation) and re-transplantation of a subject's own cells or organs. In some instances, an “autologous transplant” includes cells grown or cultured from a subject's own cells. For example, in the methods of the present disclosure, the cardiac stem cells may be derived from a cardiac tissue sample excised from the heart of the patient to be treated, cultured, engineered to be fused with platelet membrane vesicles according to the methods of the disclosure and then administered to the same patient for the treatment of a cardiovascular injury therein.

The term “allogeneic” refers to deriving from or originating in another subject or patient. An “allogeneic transplant” refers to collection (e.g., isolation) and transplantation of the cells or organs from one subject into the body of another. In some instances, an “allogeneic transplant” includes cells grown or cultured from another subject's cells.

The term “transplant” as used herein refers to cells, e.g., cardiac progenitor cells, introduced into a subject. The source of the transplanted material can include cultured cells, cells from another individual, or cells from the same individual (e.g., after the cells are cultured, enriched, or expanded ex vivo or in vitro).

The terms “treatment,” “therapy,” “amelioration” and the like refer to any reduction in the severity of symptoms. As used herein, the terms “treat” and “prevent” are not intended to be absolute terms. Treatment can refer to any delay in onset, amelioration of symptoms, improvement in patient survival, repair/regeneration of heart tissue or blood vessels, increase in survival time or rate, etc. The effect of treatment can be compared to an individual or pool of individuals not receiving the treatment. In some instances, the effect can be the same patient prior to treatment or at a different time during the course of therapy. In some aspects, the severity of disease, disorder or injury is reduced by at least 10%, as compared, e.g., to the individual before administration or to a control individual (e.g., healthy individual or an individual no longer having the disease, disorder or injury) not undergoing treatment. In some instances, the severity of disease, disorder or injury is reduced by at least 20%, 25%, 50%, 75%, 80%, or 90%. In some cases, the symptoms or severity of disease are no longer detectable using standard diagnostic techniques.

The terms “subject,” “patient,” “individual” and the like are used interchangeably and refer to, except where indicated, mammals such as humans and non-human primates, as well as rabbits, rats, mice, dogs, cats, goats, pigs, cows, and other mammalian species. The term does not necessarily indicate that the subject has been diagnosed with a particular disease, but typically refers to an individual under medical supervision.
Description

Vascular endothelium provides a barrier between subendothelial matrix and circulating cells including blood cells and platelets. It has been established that ischemic heart injures such as acute myocardial infarction (MI) can induce vascular damage and expose components of subendothelial matrix including collagen, fibronectin and von Willebrand factor (vWF) to recruit platelets. Platelets can then accumulate and bind to the injured vasculatures in MI. Such platelet recruitment is based on the matrix-binding abilities of various platelet surface molecules such as glycoprotein (GP) VI, GPIb-IX-V and gpllb/llla (Lippi et al., (2011) Nat. Rev. Cardiol. 8: 502-512). Platelets may form co-aggregates with circulating CD34⁺ progenitors in patients with acute coronary syndromes and thereby increase peripheral recruitment within the ischemic microcirculatory district and promote adhesion to the vascular lesion to promote healing (Stellos et al., (2013) Eur. Heart J. 34: 2548-2556). Cardiosphere-derived cardiac stem cells (or CSCs) have been investigated, from laboratory animal model studies (Li et al., (2012) J. Am. Coll. Cardiol. 59: 942-953; Smith et al., (2007) Circulation 115: 896-908; Cheng et al., (2010) Circ. Res. 106: 1570-1581; Lee et al., (2011) J. Am. Coll. Cardiol. 57: 455-465; Cheng et al., (2014) JACC Heart Fail. 2: 49-61) to a recently completed phase I clinical trial (Malliaras et al., (2014) J. Am. Coll. Cardiol. 63: 110-122; Makkar et al., (2012) Lancet 379: 895-904), for the treatment of MI. Like other cell types, CSCs also suffer a deficit retention rate in the heart after delivery (Cheng et al., (2014) Nat. Commun. 5: 4880).

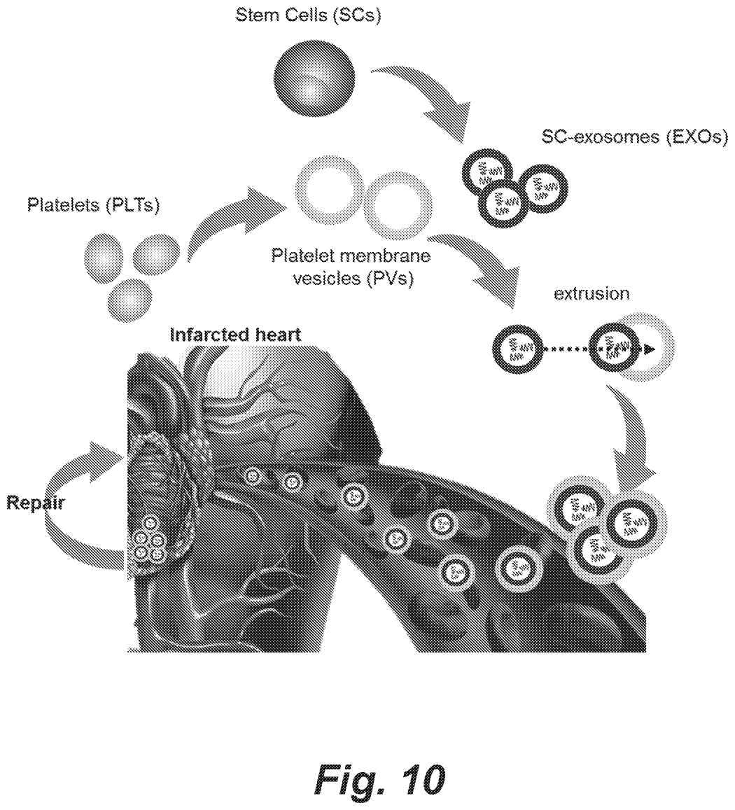

The present disclosure encompasses compositions that combine platelet-derived membrane vesicles and cells, including but not limited to stem cells, or extracellular vesicles such as exosomes derived from the cells to harness the ability of platelets to target a site of injury to a blood vessel. Stem cells, however, such as cardiac or mesenchymal stem cells offer the advantage of the ability to invade and differentiate into cell types for the repair of injured tissue. Such decoration of cells, or extracellular vesicle such as an exosomes derived from such as cells, with platelet-derived membrane vesicles is nontoxic as it does not alter the viability and functions of stem cells, or extracellular vesicles, but augments the targeting of the engineered PNV-cells/exosomes for enhanced therapeutic outcomes. Intact stem cells, or any other type of cell, can fuse with the platelet-derived membrane vesicles, whereupon the platelet membrane proteins that can bind to the vascular subendothelial matrix become integral to the membranes of the stem cells. However, with exosomes, as shown in FIG. 10, the platelet-derived membranes vesicles can encapsulate the exosomes. This allows the PNV-exosomes constructs to selectively bind to the subendothelial matrix.

The present disclosure also encompasses embodiments of a method for generating a population of engineered stem cells or extracellular vesicle such as an exosomes derived therefrom that can specifically target injury-exposed ligands or receptors of platelet-specific polypeptide markers. In particular, the methods of the disclosure are advantageous for the generation of engineered stem cells or extracellular vesicle such as an exosomes that, when administered to a subject having a tissue injury, will specifically target and bind to injured vascular tissue. Once so attached, the stem cells can migrate through the underlying subendothelial matrix into the injured tissue to differentiate and replicate to repair the site of the injury. While the methods of cell or extracellular vesicle engineering disclosed can be applied to any cell line, and in particular, to stem cells, or exosomes derived therefrom, it had been found especially advantageous to engineer cardiac stem cells or their exosomes to target the site of cardiac or cardiovascular injury.

The present disclosure encompasses the ability of platelet surface markers to selectively bind to a site of subendothelial matrix following the denuding of the endothelial cells after a vascular injury event. For example, myocardial infarction, ischemic stroke, and the like result in vascular damage that indirectly leads to injury or death to tissues normally sustained by the blood vessel. Particularly vulnerable is cardiac tissue, brain or other neural tissue. Loss of vascular endothelial cells exposes sites of the underlying matrix that allow the platelet-binding sites fused into the membranes of stem cells to selectively bind thereto to concentrate the engineered stem cells at the site of injury. The attached cells may then migrate through the matrix into the surrounding damaged tissue, whereupon the stem cells can differentiate, proliferate, and thus regenerate the lost or injured or lost tissue.

The engineered cells of the disclosure are an isolated population of cells that may be expanded by tissue culture after being isolated from a tissue of an animal or human. Most advantageously the cells are, but not limited to, stem cells such as stem cells isolated from cardiac tissue, cultured under conditions appropriate for population expansion as cardiospheres and then incubated with platelet membrane vesicles.

These membrane vesicles consist of fragments of the outer membranes of platelets and accordingly comprise a spectrum of the cell surface markers of the parent platelets and, in particular, those ligands or receptor proteins or peptides that allow platelets to bind to such as the subendothelial matrix of a blood vessel or cardiac tissue exposed by injury, for example, due to a myocardial infarction such a myocardial. The methods of generating the engineered stem cells and their use, as herein disclosed, may also be usefully applied to any suitable stem cells for the regeneration of tissues other than cardiac tissue.

The present disclosure, therefore, provides methods for the generating of populations of engineered cells or derivatives thereof targeted to a tissue injury site to be used for administration to subject having damaged or diseased tissue so as to repair, regenerate, and/or improve the anatomy and/or function of the damaged or diseased tissue. While multiple types of stem cells may be processed according to the methods disclosed herein, in several embodiments, stem cells such as, but not limited to, cardiac stem cells can be usefully generated by processing tissue and then engineered to target a site of vascular injury. The engineered cardiac stem cells, in particular, can be fused with platelet-derived membrane vesicles such that platelet-specific cell surface components are included in the outer cell membranes of the stem cells. Alternatively, extracellular vesicle such as an exosomes derived from such cells can be encapsulated by the platelet-derived membrane vesicles. Targeting the site of cardiac or blood vessel damage concentrates transplanted cardiac stem cells at the injury and increases the likelihood of establishing regeneration of damaged cardiac or cardiovascular injury.

Multiple types of cardiac stem cells can be obtained according to the methods disclosed herein including, but not limited to, cardiospheres and cardiosphere-derived cells (CDCs), ckit (CD117)-positive cells, nkx2.5-positive cardiac cells, and the like. Embodiments of the engineered cardiac stem cells of the present disclosure are advantageous for the treatment or repair of damaged or diseased cardiac tissue that may have resulted from one or more of acute heart failure (e.g., a stroke or myocardial infarction) or chronic heart failure (e.g., congestive heart failure). In some embodiments of the methods of the disclosure, about 1×10⁵ to about 1×10⁷ of cardiac stem cells may be administered. The dose can be varied depending on the size and/or age of a subject receiving the cells. Different routes of administration are also used, depending on the embodiment. For example, the cardiac stem cells may be administered by intravenous, intra-arterial, intracoronary, or intramyocardial routes of administration.

Provided herein, therefore, are methods of treating a human subject with an injured heart by administering engineered cardiac stem cells, as described herein. In some embodiments, the engineered cardiac stem cells may be administered to a patient with acute myocardial infarction or having myocardial ischemia. Further provided are methods of treating a patient in need of angiogenesis (e.g., growth of blood vessels) by administering the engineered cardiac stem cells of the disclosure. The engineered cardiac stem cells of the disclosure can be used to ameliorate the effects of any type of injury to the heart.

Cardiac stem cell therapy (and the methods disclosed herein) may be autologous, allogeneic, syngeneic, or xenogeneic, depending on the needs of the subject patient. In several embodiments, allogeneic therapy is employed, as the ready availability of tissue sources (e.g., organ donors, etc.) enables a scaled-up production of a large quantity of cells that can be stored and subsequently used in an “off the shelf” fashion. Preferably, the source of both the stem cells (or extracellular vesicle such as an exosomes thereof) and the platelets that provide the membrane vesicles is the subject patient that receives the engineered cells or extracellular vesicles to reduce the possibility of adverse immunological reactions. However, it may also be advantageous to use stored stem cells and platelet-rich plasma derived from another person when the subject patient requires treatment earlier than the time required to culture his/her own cardiac stem cells for fusion with platelet membrane vesicles.

Stem cell transplantation is currently implemented clinically but is limited by low retention and engraftment of transplanted cells. Platelets play an important role in recruiting stem cells to the sites of injury on blood vessels. To harness the natural injury-homing ability of platelets to enhance the vascular delivery of cardiac stem cells (CSCs) to myocardial infarction (MI) injury, platelet nanovesicles (PNVs) were fused onto the surface of isolated CSCs to form engineered PNV-CSCs. Cell proliferation, migration and viability of CSCs are not compromised by this PNV decorating.

PNV-CSCs of the disclosure possessed surface markers of platelets, which are associated with platelet adhesion to injury sites. In vitro, it has been shown that the PNV-CSCs can selectively bind to a collagen-coated surface and denuded rat aorta. In a rat model of acute MI, PNV decoration increases CSC retention in the heart and augments therapeutic benefits.

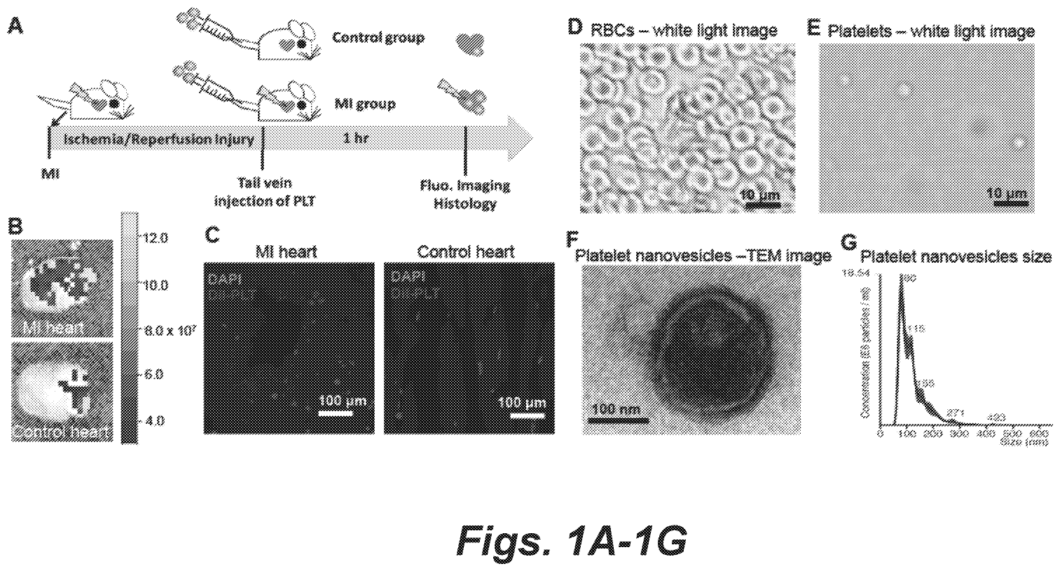

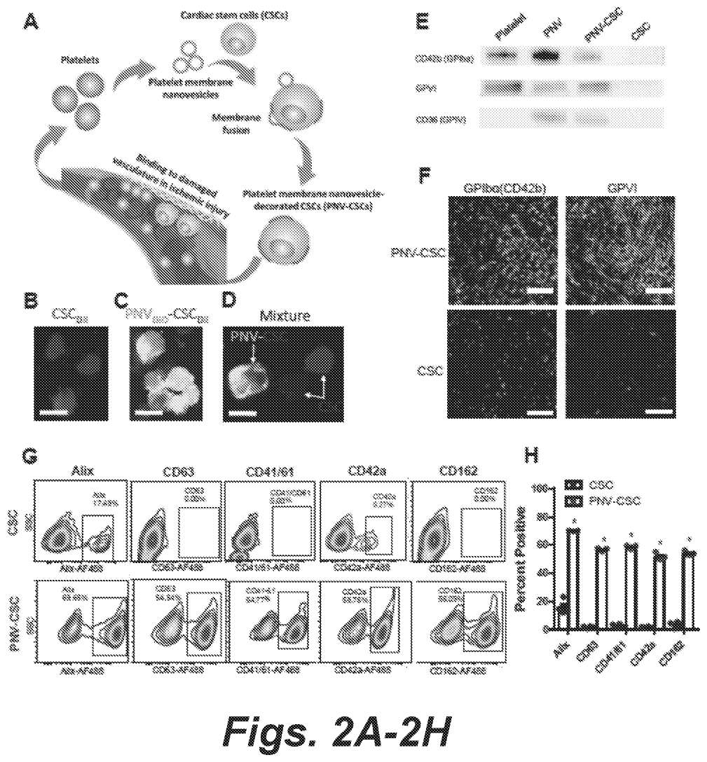

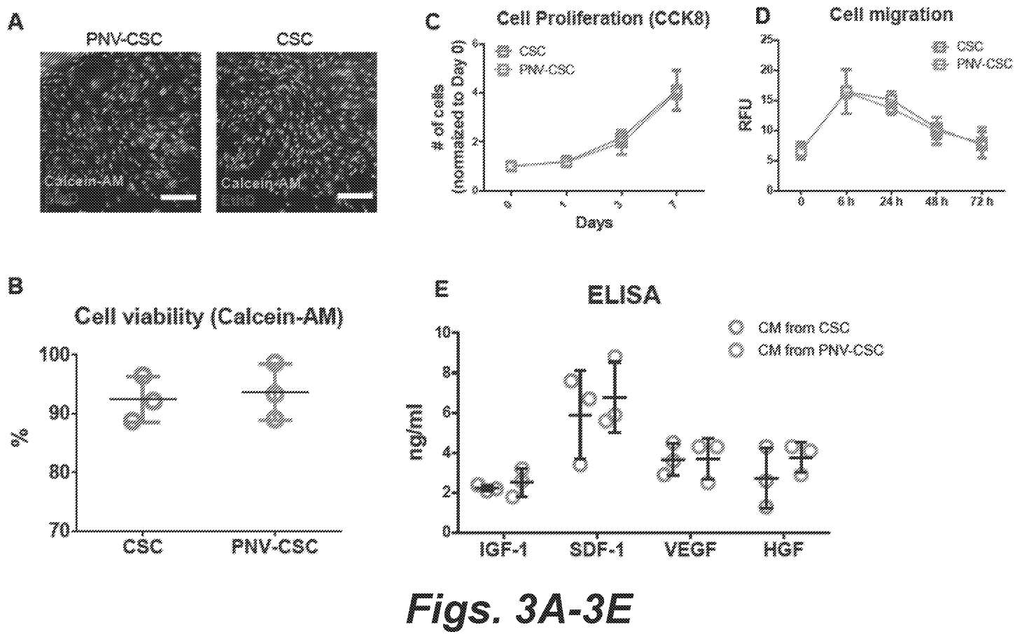

PNV-CSCs treatment robustly boosts cardiac function with the highest left ventricular ejection fraction and best cardiac morphology by promoting angiomyogenesis. The engineered PNV-CSCs possess the natural targeting and repairing ability of their parental cells: i.e. of both platelets and of CSCs. This method advantageously offers a method of stem cell manipulation that is without obvious side-effects, with no genetic alterations of the cells, and is generalizable to multiple cell types.
Intravenously injected platelets target myocardial infarction: To evaluate the natural MI-homing ability of platelets, CM-DiI labeled platelets were intravenously injected through the tail veins of animals with recent ischemia/reperfusion-induced MI (as shown in FIG. 1A). Ex vivo fluorescent imaging at 1 h after injection revealed a larger amount of injected platelets were retained in the MI heart as compared to normal heart (i.e. no MI) (FIG. 1B). Histology further confirmed platelets concentrated at the myocardium injury region (FIG. 1C). These compound results confirmed the MI-homing ability of platelet and indicated the potential of targeting platelet membrane vesicle-engineered stem cells to MI.
Characterization of platelet membrane vesicles: To facilitate membrane fusion with CSCs, platelet nanovesicles were derived from intact platelets. Bright field images indicated the distinctive morphologies of red blood cells (FIG. 1D) and platelets (FIG. 1E). Transmission electron micrograph showed the morphology of platelet nanovesicles (FIG. 1F). Nanosight® analysis revealed the size distribution of platelet nanovesicles (FIG. 1G) with an average size around 100 nm.
Characterization of platelet nanovesicle-decorated cardiac stem cells (PNV-CSCs): Platelet nanovesicles (PNV) were derived from platelets and decorated onto the surface of CSCs to form platelet nanovesicle-decorated cardiac stem cells (PNV-CSCs) through membrane fusion facilitated by co-incubation in PEG (FIG. 2A). Fluorescent microscopic imaging showed CM-DiI pre-labeled CSC (FIG. 2B) were decorated with green fluorescent DiO pre-labeled platelet membrane vesicles to form PNV-CSC (FIG. 2C). To demonstrate that the fluorescence overlapping is not simply from dye transfer, PNV-CSCs were co-cultured with control CSCs for 24 hrs. No evident dye transfer from PNV-CSCs to CSCs was noticed (FIG. 2D). Western blotting further showed distinct platelet surface markers including CD42b (GPIba), GPVI and CD36 (GPIV) expression in platelets, platelet-vesicles, and PNV-CSCs, but not in control CSCs (FIG. 2E). Immunocytochemistry staining confirmed CD42b (GPIbα) and GPVI expressions on PNV-CSCs (FIG. 2F, upper panels) but not on control CSCs (FIG. 2F, lower panels). Flow cytometry confirmed the expressions of platelet surface markers on PNV-CSCs, as shown in FIGS. 2G and 2H. These compound datasets indicated PNV fusion onto CSCs. Expression of primary platelet membrane proteins/protein subunits on PNV-CSCs indicated the engineered PNV-CSC incorporated binding motifs of the platelets.
Platelet nanovesicle decoration does not affect PNV-CSCs' viability and functions: To further determine if the platelet nanovesicle decoration of the present disclosure would affect the viability and function of PNV-CSCs, a Live/Dead assay was performed on PNV-CSCs or CSCs tissue plate cultured plates 7 days (FIG. 3A). Pooled data indicated comparable cell viabilities of PNV-CSCs and CSCs (FIG. 3B). A CCK-8 assay showed that the proliferation rates of PNV-CSCs or CSCs were indistinguishable (FIG. 3C). Trans-well migration assay showed PNV-CSCs or CSCs had similar migration potencies at various time points (FIG. 3D).

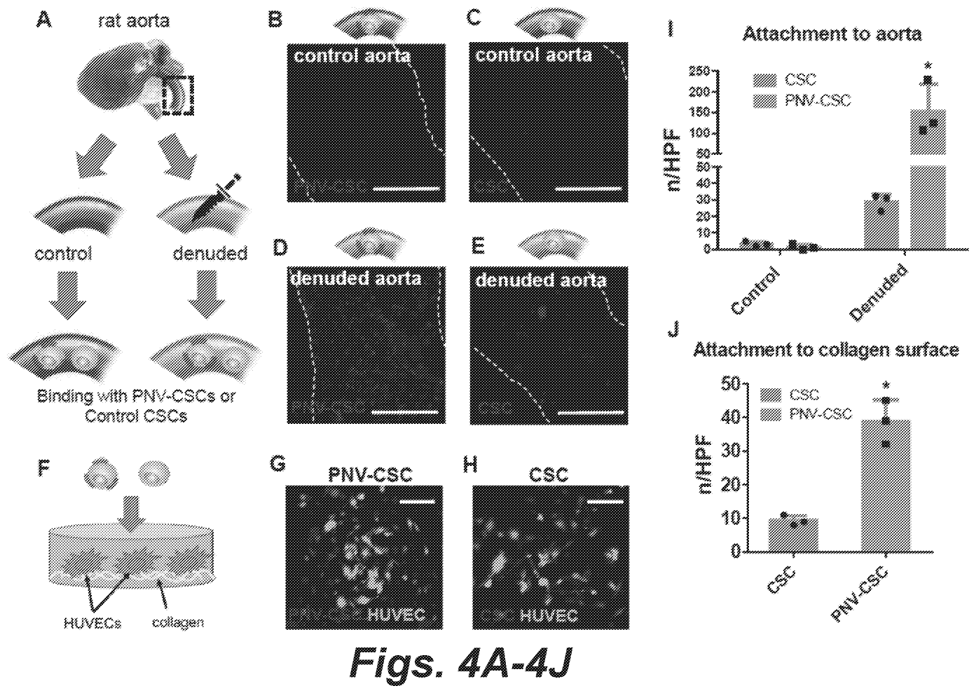

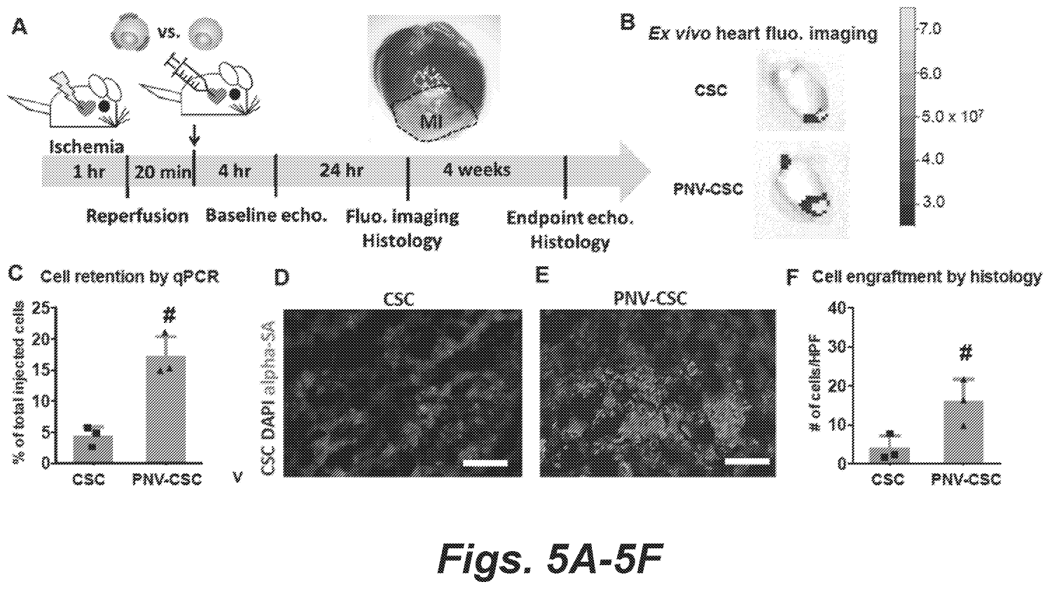

A major mode of action of injected stem cells, the releasing of growth factors including insulin-like growth factor (IGF)-1, stromal cell-derived factor (SDF)-1, vascular endothelial growth factor (VEGF) and hepatocyte growth factor (HGF) from PNV-CSCs, was not undermined by platelet nanovesicle decoration (FIG. 3E). These data suggested PNV decoration was nontoxic to CSCs and could retain the regenerative potency of original CSCs.
Binding of PNV-CSCs to collagen surface and denuded aorta in vitro: The binding potency of PNV-CSCs ex vivo in excised damaged vasculatures was tested. A segment of rat aorta was obtained and surgically scraped to expose the subendothelial matrix (FIG. 4A). Microscopic imaging showed no binding of DiI-labeled PNV-CSCs or CSCs on control (non-denuded) aortas (FIGS. 4B and 4C). PNV-CSCs had robust binding to denuded aorta (FIG. 4D) while control CSCs did not (FIG. 4E). Quantitative analysis confirmed PNV-CSCs' binding to denuded aortas with greater specificity and sensitivity compared to control CSCs (FIG. 4I). Furthermore, DiI-labeled PNV-CSCs or CSCs were plated onto GFP-tagged human umbilical vein endothelial cells (HUVECs) cultured on collagen-coated surfaces (FIG. 4F). Augmented adherence of PNV-CSCs was found (FIGS. 4G-4J).
PNV-CSCs exhibited superior retention/engraftment in rats with ischemia/reperfusion injury: To test the therapeutic potential of PNV-CSCs, a rat model of ischemia/reperfusion by temporary LAD ligation for 1 hour (FIG. 5A) followed by reperfusion was employed. After 20 mins of reperfusion, 5×10⁵ PNV-CSCs or control CSCs were intracoronary-injected. Cells were injected into the LV cavity during temporary occlusions of the aorta. These cells perfused into the myocardium through the coronary arteries in the closed circuit which mimicked intracoronary injection.

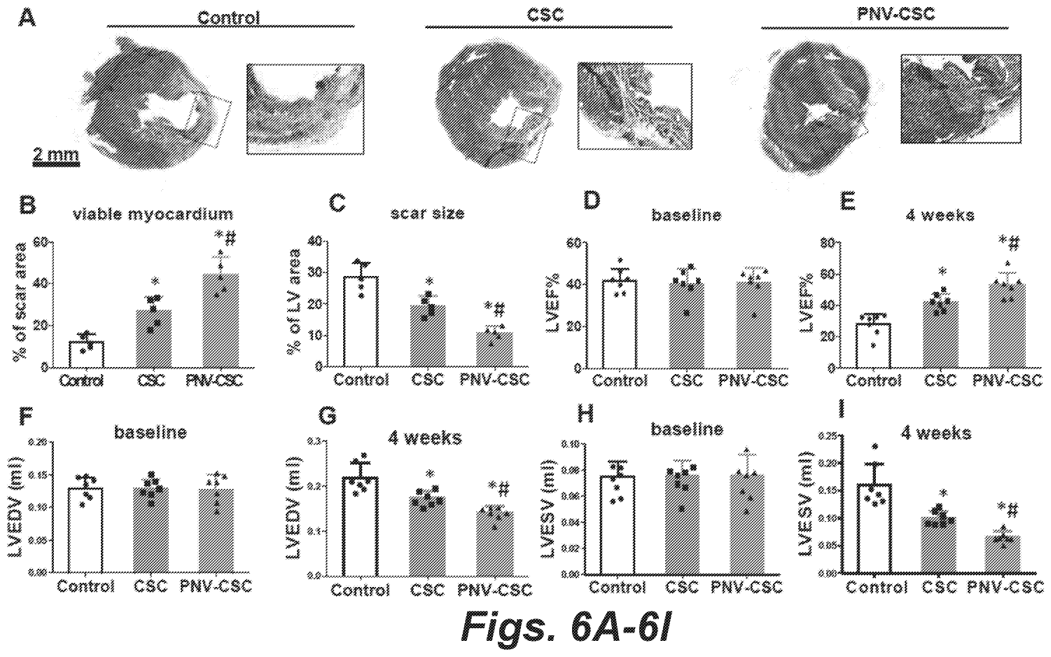

Ex vivo fluorescent imaging at 24 h showed that PNV decoration boosted CSC retention in the heart (FIG. 5B). This was further confirmed by qPCR analysis. A cohort of female rats received PNV-CSCs or CSCs from male donors to quantitation of cell retention by SRY qPCR (FIG. 5C). Furthermore, IHC on heart sections revealed superior engraftment of PNV-CSCs (red, FIG. 5E) over CSCs (red, FIG. 5D) in the post-MI heart. Quantitative analysis confirmed significant retention of PNV-CSCs in the heart (FIG. 5F). Thus, PNV-CSCs exhibited greater retention/engraftment in rats with ischemia/reperfusion injury.
Augmented functional benefits from PNV-CSCs therapy: To investigate whether augmented cell retention translates into augmentation of therapeutic benefits, cardiac morphology, fibrosis, and pump function were evaluated. Masson's trichrome-staining 4 weeks after treatment (FIG. 6A) showed apparent heart morphology protection by CSCs as compared to control-injected hearts, consistent with published results (Cheng et al., (2012) Cell Transplant 21:1121-1135; Kanazawa et al., (2015) Circ. Heart Fail 8: 322-332). The greatest protective effects were seen in the PNV-CSCs-treated hearts with the highest amount of viable myocardium (FIG. 6B) but the smallest scar size (FIG. 6C). Left ventricular ejection fractions (LVEFs) were measured at baseline (4 h post-infarct) and 4 weeks afterwards. LVEFs were indistinguishable at baseline for all three groups (FIG. 6D), indicating a similar degree of initial injury. Over the 4 weeks period, the LVEFs in control treated animals continued to deteriorate (FIG. 6E) while the CSCs-treated animals exhibited a trend of LVEF preservation (FIG. 6E), but PNV-CSCs treatment robustly boosted cardiac function with the highest LVEFs at 4 weeks (FIG. 6E). Left ventricular end diastolic volume (LVEDV) (FIGS. 6F and 6G) and left ventricular end systolic volume (LVESV) (FIGS. 6H and 6I) were also measured. In both measures, PNV-CSC treatment generated a larger protective effects than CSC-alone treatment.

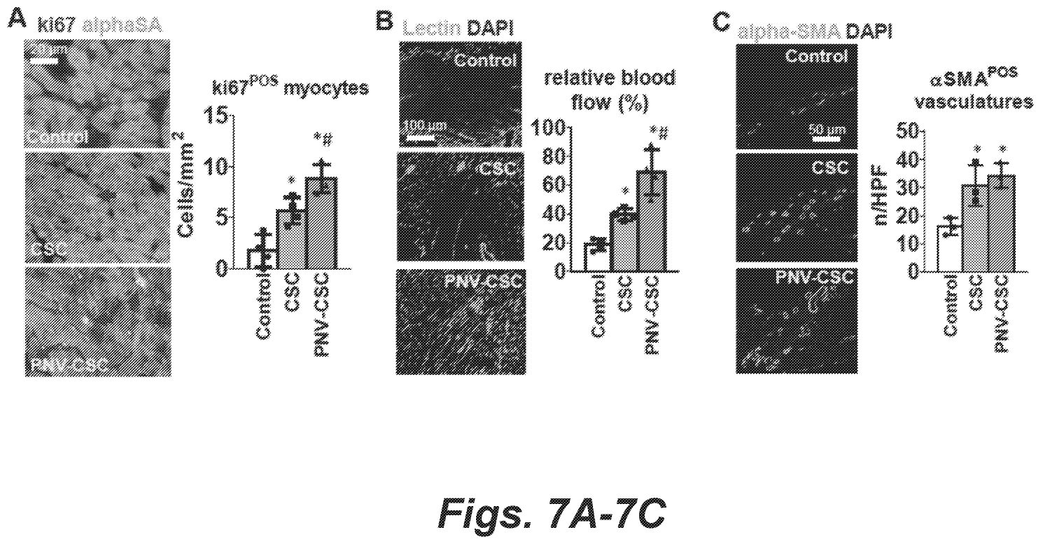

To investigate the mechanisms underlying the therapeutic benefits of PNV-CSCs, a series of immunohistochemistry to the treated hearts was performed. The results suggested PNV-CSC treatment led to increased cardiomyocyte cycling (FIG. 7A) and relative blood flow by lectin angiogram (FIG. 7B). Futhermore, PNV-CSC and CSC treatmet increased vessel density as compared to control treatment (FIG. 7C).

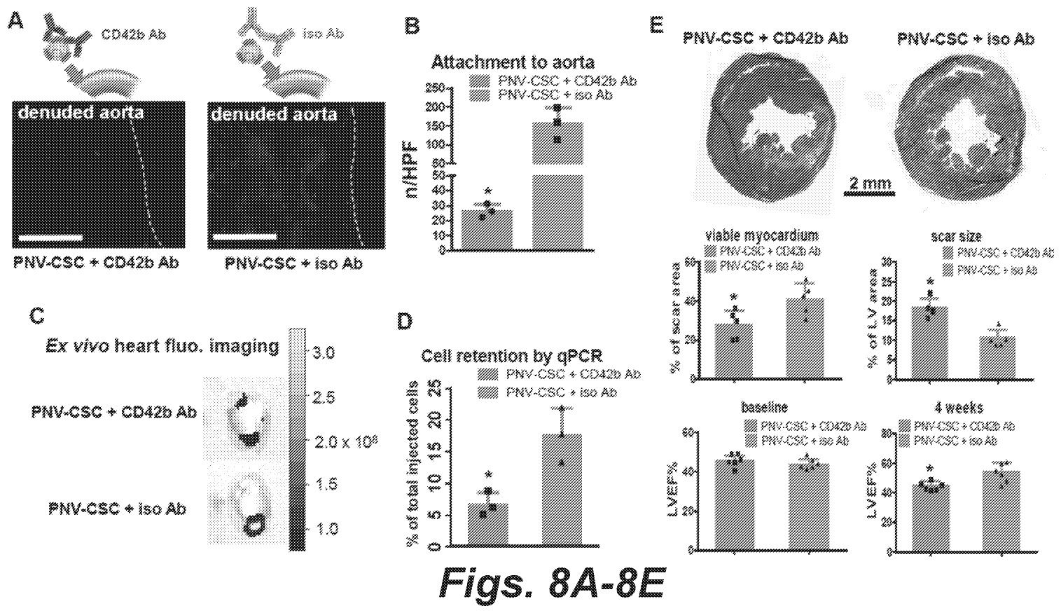

To further explore the adhesion molecules involved in the targeting PNV-CSCs to MI, anti-CD42b neutralizing antibodies or isotype control antibodies were used to pretreat PNV-CSCs before the experiments. CD42b inhibition blunted the ability of PNV-CSCs to bind denuded rat aorta (FIGS. 8A and 8B), reduced the retention of PNV-CSCs in the heart (FIGS. 8C and 8D), and ultimately diminished the therapeutic potency of PNV-CSCs in the same rat model of I/R injury (FIG. 8E). These results show that CD42b played an essential role in homing PNV-CSCs to MI injury.

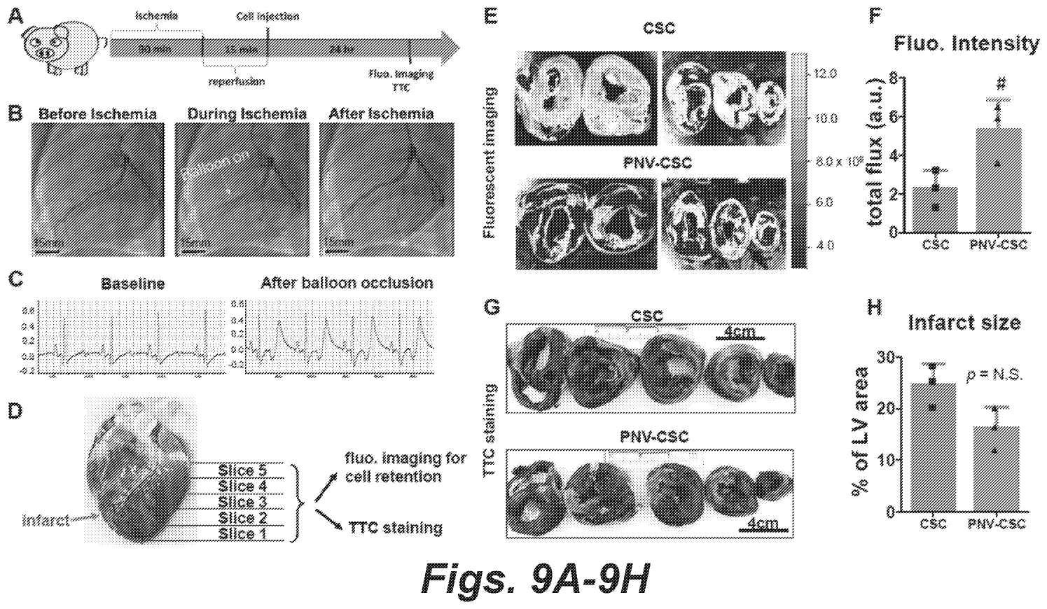

The findings were translated into a pig model of acute MI. Acute MI was created in farm pigs with an ischemia-reperfusion model by balloon occlusion (FIGS. 9A-9D). Fluorescent imaging confirmed the superior cardiac retention of PNV-CSCs over that of CSCs, as shown in (FIGS. 9E and 9F). TTC staining conferred that PNV-CSC treatment showed little or no adverse effects and did not exacerbate infarct size (FIGS. 9G and 9H).

Accordingly, the natural injury-targeting power of platelets have been used to enhance the vascular delivery and therapeutic outcome of cardiosphere-derived stem cells (CSCs). The data show that: (i) PNV decoration was effective and did not affect the viability and functions of CSCs in vitro; (ii) PNV decoration enhanced CSC binding to denuded (injured) blood vessel ex vivo; (iii) in a rat model of ischemia/reperfusion, PNV decoration enhanced the targeting of CSCs to MI and augmented their ability to preserve cardiac pump functions and reduce infarct sizes; and (iv) the targeting effects of PNV decoration was confirmed in a pig model of acute MI.

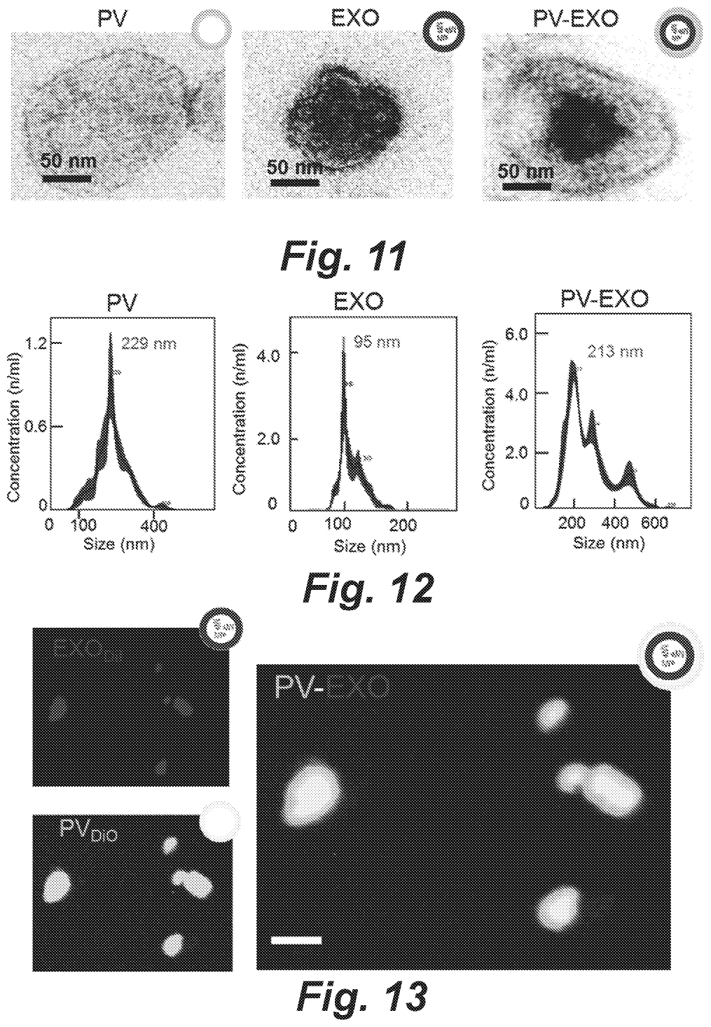

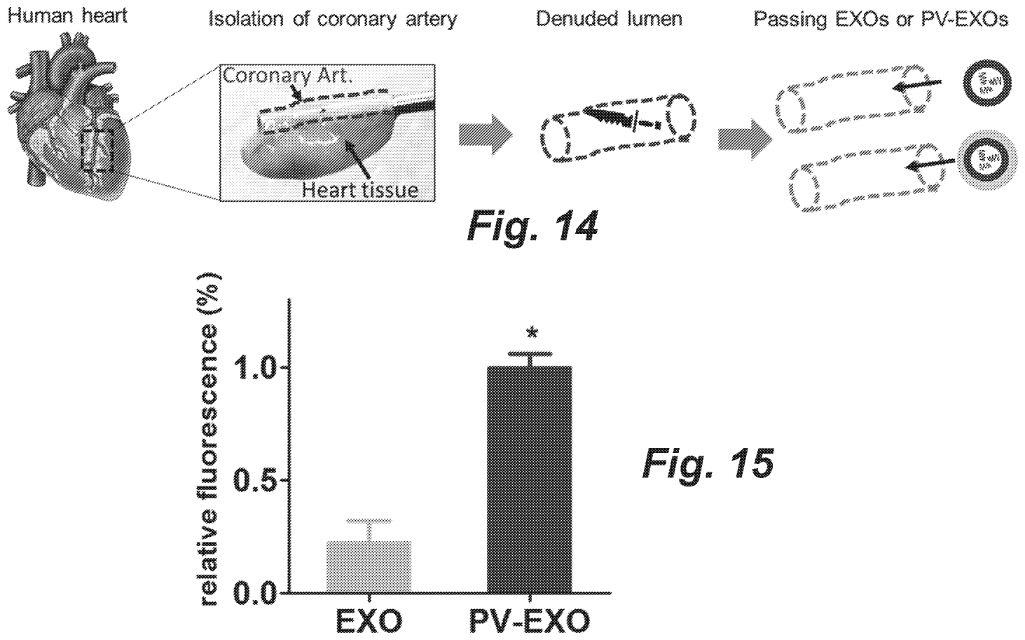

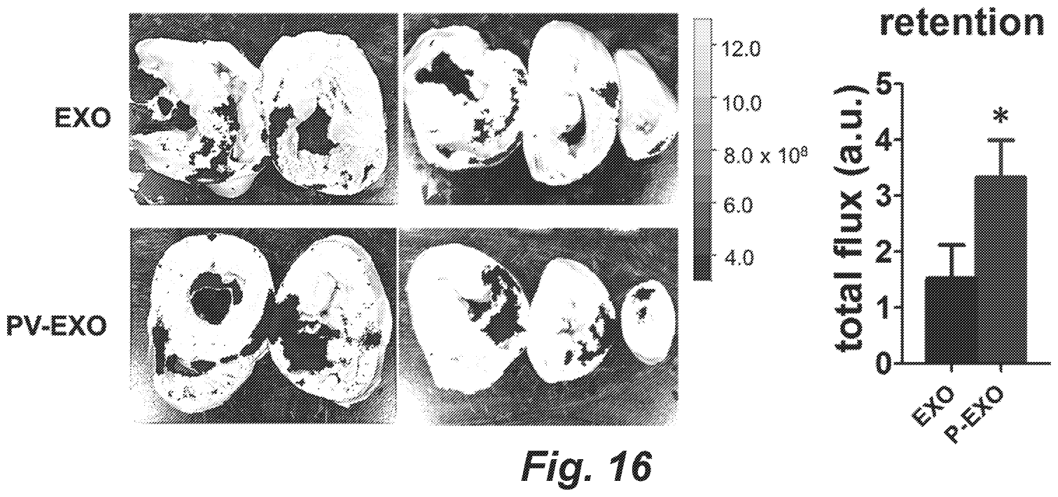

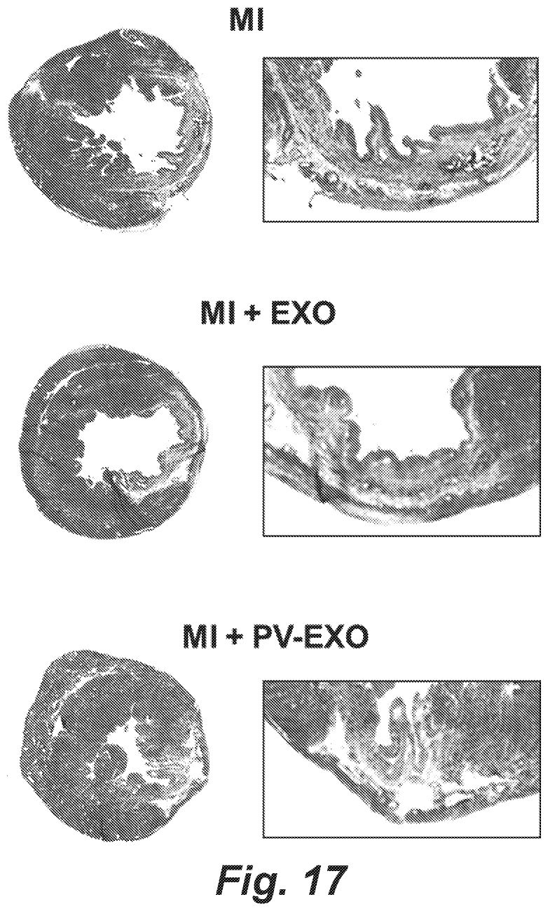

Along with the methods of the disclosure to decorate stem cells with platelet membrane vesicles, methods to incorporate platelet binding molecules onto exosomes we sought to develop. In this regard, injury-targeted exosomes were created. FIG. 10 indicates the method we employed to coat exosomes with platelet vesicles. The resulting new entity platelet vesicle-encapsulated exosome (PV-EXO) demonstrated an exosome core and a platelet vesicle coat (FIGS. 11-13). Compared to control exosomes, PV-EXO demonstrated increased binding ability to injured human blood vessels (FIGS. 14 and 15), increased targeting ability in pigs with MI (FIG. 16), and therapeutic benefits in rats with acute MI (FIG. 17).

The engineered cells, such as cardiac stem cells, or engineered extracellular vesicles such as an exosomes, of the disclosure can be administered back to the individual from which the cells were derived or to a different individual. Thus, engineered stem cells such as the engineered cardiac stem cells of the disclosure can be used for autologous or allogeneic transplantation to treat an individual with an injured heart or an individual who can benefit from angiogenesis. Engineered stem cells including, but not limited to, cardiac stem cells of the disclosure, as described herein, are non-transgenic. Therefore, they are more suitable for transplantation into a patient compared to genetically modified cells or cells transduced with viruses and the like.

In some embodiments, provided herein are methods comprising administering engineered cardiac stem cells of the disclosure to an individual who has damaged blood vessels. In some instances, the damaged blood vessels are due to disease or conditions such as peripheral arterial disease, critical limb ischemia, or chronic wounds (e.g., diabetic lower extremity ulcers, venous leg ulcers, pressure ulcers, arterial ulcers), to name a few. For example, engineered cardiac stem cells of the disclosure can be administered to an individual suffering from a stroke (e.g., acute or chronic) or a condition causing blood vessel injury in the brain in order to repair the brain.

To repair and/or regenerate blood vessels, engineered cardiac stem cells of the disclosure can be administered alone or in combination with angiogenesis-promoting factors including, but not limited to, IL-15, FGF, VEGF, angiopoietin (e.g., Ang1, Ang2), PDGF, and TGF-β.

Methods of administration include injection, transplantation, or other clinical methods of getting cells to a site of injury in the body. Non-limiting examples of injection methods that can be used to administer engineered cardiac stem cells of the disclosure include intravenous injection, intracoronary injection, transmyocardial injection, epicardial injection, direct endocardial injection, catheter-based transendocardial injection, transvenous injection into coronary veins, intrapericardial delivery, or combinations thereof.

The injection of engineered cells or extracellular vesicles of the disclosure can be either in a bolus or in an infusion. The engineered cells or extracellular vesicles of the disclosure can be combined with a pharmaceutical carrier suitable for administering to a recipient subject. Pharmaceutically acceptable carriers are determined in part by the particular method used to administer the cell composition, but are typically isotonic, buffered saline solutions. Accordingly, there are a wide variety of suitable formulations of pharmaceutical compositions for the presently described compositions (see, e.g., Remington's Pharmaceutical Sciences, 17th ed., 1989). The engineered cardiac stem cells of the disclosure as described herein can be administered in a single dose, a plurality of doses, or on a regular basis (e.g., daily) for a period of time (e.g., 2, 3, 4, 5, 6, 7, days, weeks, months, or as long as the condition persists).

The dose (e.g., the amount of cells) administered to the subject in the context of the present disclosure should be sufficient to affect a beneficial response in the subject over time, e.g., repair or regeneration of heart tissue, repair of regeneration of blood vessels, or a combination thereof. The optimal dose level for any patient will depend on a variety of factors including the efficacy of the specific modulator employed, the age, body weight, physical activity, and diet of the patient, on a possible combination with other drugs, and on the severity of the cardiac or angiogenic injury. The size of the dose also will be determined by the presence, existence, nature, and extent of any adverse side-effects that accompany the administration of the engineered cardiac stem cells in a particular subject.

The engineered cardiac stem cells of the disclosure can be transplanted into the individual at a single or multiple sites. Engineered cardiac stem cells of the disclosure can be administered alone or in combination with biomaterials (e.g., hydrogel or 3-dimensional scaffolds) prior to transplantation in order to promote engraftment and stimulate tissue repair. Engineered cardiac stem cells of the disclosure can be embedded in a biodegradable or biocompatible material that is applied to the site in need of cell-based therapy. Scaffolds can increase the retention of the engineered cardiac stem cells and the viability of the cells upon delivery to the site of injury.

Accordingly, one aspect of the disclosure encompasses embodiments of a composition comprising: (a) a platelet membrane-derived vesicle or a fragment thereof; and (b) an animal or human cell, a plurality of said cells, or an extracellular vesicle derived from the cell, wherein the platelet-derived membrane vesicle or fragments thereof can be fused into the outer membrane of the cell or plurality of said cells or encapsulates the extracellular vesicle, and wherein the composition can be characterized as having specific binding affinity for at least one component of a vascular subendothelial matrix or vascular cell.

In some embodiments of this aspect of the disclosure, the extracellular vesicle can be an exosome.

In some embodiments of this aspect of the disclosure, the animal or human cell can be a stem cell.

In some embodiments of this aspect of the disclosure, the stem cell can be a cardiac stem cell or a mesenchymal stem cell.

In some embodiments of this aspect of the disclosure, the animal or human cell can have an outer membrane engineered to have platelet-derived polypeptide cell-surface markers.

In some embodiments of this aspect of the disclosure, the animal or human cell can be isolated from an animal or human tissue, a cultured cell, or a cryopreserved cell.

In some embodiments of this aspect of the disclosure, the stem cell can be from a cultured cardiosphere from a cardiac tissue.

In some embodiments of this aspect of the disclosure, the extracellular vesicle can be derived from a cardiac stem cell or a mesenchymal stem cell.

In some embodiments of this aspect of the disclosure, the animal or human cell, plurality of cells, or the extracellular vesicle can be derived from the same animal or human subject as the platelet-derived membrane vesicle.

In some embodiments of this aspect of the disclosure, the animal or human cell, plurality of cells, or the extracellular vesicle can be derived from a different animal or human subject as the platelet-derived membrane vesicle.

In some embodiments of this aspect of the disclosure, (i) the animal or human cell, plurality of cells, or the extracellular vesicle and (ii) the platelet-derived membrane vesicle of the composition can both be derived from the animal or human subject that is a recipient of the composition for treatment of a vascular injury.

In some embodiments of this aspect of the disclosure, at least one of (a) the animal or human cell, plurality of cells, or the extracellular vesicle and (b) the platelet-derived membrane vesicle or fragments thereof of the composition can be derived from the animal or human subject that is a recipient of the composition for treatment of a vascular injury.

In some embodiments of this aspect of the disclosure, the composition can be admixed with a pharmaceutically acceptable carrier.

Another aspect of the disclosure encompasses embodiments of a method of generating a population of engineered animal or human cells or extracellular vesicles derived from said cells, the method comprising the step of mixing a population of platelet-derived membrane vesicles or fragments thereof and a population of cells or extracellular vesicles and thereby fusing the platelet-derived membrane vesicles with the outer membranes of the cells or encapsulating the extracellular vesicles, wherein said cells or extracellular vesicles can be isolated from an animal or human tissue or biofluid, cultured cells, or cryopreserved cells.

In some embodiments of this aspect of the disclosure, the method can further comprise the steps of: (i) obtaining a suspension of platelets isolated from the plasma of an animal or human subject; and (ii) sonicating the suspension of platelets to generate a population of platelet-derived membrane vesicles or fragments thereof.

In some embodiments of this aspect of the disclosure, the method can further comprise the step of incubating the cells, or extracellular vesicles derived therefrom, together with the platelet-derived membrane vesicles in the presence of polyethylene glycol (PEG) or extruding the cells, or extracellular vesicles derived therefrom, together with the platelet-derived membrane vesicles or fragments thereof, and thereby fusing the platelet-derived membrane vesicles or fragments thereof with the outer membranes of the cells or encapsulating the extracellular vesicles.

In some embodiments of this aspect of the disclosure, the extracellular vesicle can be an exosome.

In some embodiments of this aspect of the disclosure, the animal or human cells can be stem cells.

In some embodiments of this aspect of the disclosure, the stem cells can be derived from a cardiac tissue.

In some embodiments of this aspect of the disclosure, the method can further comprise the step of obtaining the stem cells from a cultured tissue explant derived from a cardiac tissue.

In some embodiments of this aspect of the disclosure, the method can further comprise the step of obtaining the platelet-derived membrane vesicles or fragments thereof and the cells, or the extracellular vesicles derived from said cells, from the same animal or human subject.

In some embodiments of this aspect of the disclosure, the method can further comprise the step of obtaining the platelet-derived membrane vesicles or fragments thereof and the cells, or the extracellular vesicles derived from said cells, from different individual animal or human subjects.

Yet another aspect of the disclosure encompasses embodiments of a method of repairing a tissue injury in an animal or human subject, the method comprising administering to a recipient animal or human patient having a tissue injury a composition comprising a population of engineered cells or extracellular vesicles derived from said cells, wherein the engineered cells or extracellular vesicles comprise a platelet-derived membrane vesicle or fragments thereof fused into the outer membrane of the cell or encapsulating the extracellular vesicle or vesicles, and wherein the engineered cells or extracellular vesicles selectively target the subendothelial matrix or a vascular cell at the site of the tissue injury when administered to a recipient animal or human.

In some embodiments of this aspect of the disclosure, the engineered cells can be engineered stem cells or extracellular vesicles derived from said stem cells and the tissue injury of the subject can be to a tissue of the cardiovascular system.

In some embodiments of this aspect of the disclosure, the engineered cells can be cardiac or mesenchymal stem cells and the extracellular vesicles can be derived from said cardiac or mesenchymal stem.

In some embodiments of this aspect of the disclosure, the extracellular vesicles can be exosomes.

In some embodiments of this aspect of the disclosure, the tissue injury can be accompanied by an injury to a blood vessel.

In some embodiments of this aspect of the disclosure, the tissue injury can be of neural tissue, muscular tissue, cardiac tissue, or hepatic tissue, and wherein the engineered cells migrate to the injured tissue.

In some embodiments of this aspect of the disclosure, the engineered cells or engineered extracellular vesicles can be derived from the same animal or human subject as the platelet-derived membrane vesicles or fragments thereof.

In some embodiments of this aspect of the disclosure, the engineered cells or engineered extracellular vesicles are not derived from the same animal or human subject as the platelet-derived membrane vesicles or fragments thereof.

In some embodiments of this aspect of the disclosure, at least one of (i) the engineered cells or engineered extracellular vesicles derived therefrom and (ii) the platelet-derived membrane vesicles or fragments thereof, are derived from the recipient animal or human patient.

It should be emphasized that the embodiments of the present disclosure, particularly any “advantageous” embodiments, are merely possible examples of the implementations, merely set forth for a clear understanding of the principles of the disclosure. Many variations and modifications may be made to the above-described embodiment(s) of the disclosure without departing substantially from the spirit and principles of the disclosure. All such modifications and variations are intended to be included herein within the scope of this disclosure, and protected by the following claims.

The specific examples below are to be construed as merely illustrative, and not limitative of the remainder of the disclosure in any way whatsoever. Without further elaboration, it is believed that one skilled in the art can, based on the description herein, utilize the present disclosure to its fullest extent. All publications recited herein are hereby incorporated by reference in their entirety.

The following examples are put forth so as to provide those of ordinary skill in the art with a complete disclosure and description of how to perform the methods and use the compositions and compounds disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. The term “about” can include ±1%, ±2%, ±3%, ±4%, ±5%, ±6%, ±7%, ±8%, ±9%, or ±10%, or more of the numerical value(s) being modified.

### EXAMPLES

### Example 1

Isolation of platelets and generation of platelet nanovesicle: Isolation of platelets and generation of platelet nanovesicle were performed as previously described by Hu et al., (2015) Nature 526:118-21 (2015), incorporated herein by reference in its entirety. Briefly, blood from WKY male rats was collected into an EDTA tube and then centrifuged at 100 g for 20 mins at room temperature to separate red blood cells and white blood cells. The collected supernatant containing platelets (i.e., platelet rich plasma, or PRP) was further centrifuged at 100 g for 20 min to remove remaining blood cells. PBS with 1 mM of EDTA and 2 mM of Prostaglandin E1 (PGE1, Sigma Aldrich, MO, USA) was added to purified PRP to prevent platelet activation.

Platelets were pelleted by centrifugation at 800 g for 20 min at room temperature, after which the supernatant was discarded and the platelets were resuspended in PBS containing 1 mM of EDTA and mixed with protease inhibitor (Thermo Fisher Scientific, MA, USA). Platelets were aliquoted into 1 ml samples and placed in −80° C. before usage. After a repeated freeze-thaw process, platelet samples were thawed to room temperature and centrifuged at 4,000 g for 3 minutes. After three repeated washes with PBS and mixture with protease inhibitors, the pelleted platelets were suspended in water and sonicated in a capped glass vial for 5 min using a Fisher Scientific FS30D bath sonicator at a frequency of 42 kHz and a power of 100 W. The presence of platelet membrane vesicles was verified for size distribution using a NanoSight and morphological features using transmission electron microscopy (TEM).
Derivation and culture of rat CSCs: Rat CSCs were derived from the hearts of WKY rats before using cardiosphere method as described in Cheng et al., (2010) Circ. Res. 106:1570-1581 (2010); Vandergriff et al., (2014) Biomaterials 35:8528-8539; Li et al., (2010) Stem Cells. 28: 2088-2098, incorporated herein by reference in their entireties.

Briefly, hearts were minced into fragments less than 2 mm³, then washed with PBS and partially digested with collagenase (Sigma Aldrich). The tissue fragments were cultured as cardiac explants on 0.5 mg/ml fibronectin solution-coated surfaces in Iscove's Modified Dulbecco's Medium (IMDM; Thermo Fisher Scientific) containing 20% fetal bovine serum (FBS). After that, a layer of stromal-like cells emerged from the cardiac explant with phase bright cells over them. The explant-derived cells were harvested using TryPEL Select (under direct visualization or no more than 5 minutes, GIBCO). Harvest cells were seeded at a density of 2×10⁴ cells/ml in UltraLow Attachment flaks (Corning) for cardiosphere formation. In about 3-7 days, explant-derived cells spontaneously aggregated into cardiospheres. The cardiospheres were collected and plated onto fibronectin-coated surface to generate cardiosphere-derived cardiac stem cells (CSCs). The culture was maintained in IMDM (Thermo Fisher Scientific) containing 20% FBS.
Generation and characterization of platelet nanovesicle-decorated cardiac stem cells (PNV-CSCs): Platelet nanovesicle decoration on CSCs (PNV-CSCs) was performed by mixing cells in presence of polyethylene glycol (PEG). Briefly, 1×10⁷DiI-labeled CSCs pellet and 1×10¹⁰ of DiO-labeled PNVs were mixed in 50 μl PEG for 5 mins. The suspension was then diluted by 10 ml warm serum-free medium and treated cells were retrieved by centrifugation at 410 rcf for 5 mins (Li et al., (2015) Biomaterials 54: 177-187; Kawada et al., (2003) Int. J. Cancer 105: 520-526; Lentz, B. R. (1994) Chem. Phys. Lipids. 73: 91-106). Cell proliferation, viability and migration of PNV-CSCs were characterized and compared to control CSCs. Cell Counting Kit-8 (Dojindo Molecular Technologies, MD, USA) was used to quantify cellular proliferation at Day 0, 1, 3 and 5. The absorbance rate was read by a microplate reader (Tecan Sunrise, Switzerland). For cell viability, PNV-CSCs or CSCs were cultured on TCP for 7 days and then stained with LIVE/DEAD® Viability/Cytotoxicity Kit (Thermo Fisher Scientific) and live cell number in 3 randomized selected microscopic field were counted. A transwell plate setup allowed for cell migration through pores into the lower chamber where they could be detected. Fluorescently-labeled PNV-CSCs or CSCs were incorporated and FBS served as a chemoattractant in the lower chamber. When the PNV-CSCs or CSCs migrated from the upper to the lower chamber, fluorescence (RFU) increased. Secretion of growth factors including IGF-1, SDF-1, VEGF and HGF by PNV-CSCs and CSCs were determined by ELISA kits (R&D Systems, MN, USA).
Examination of platelet-specific surface markers on PNV-CSCs: To further confirm successful membrane fusion, western blot analysis was performed on PNV-CSCs using antibodies against major platelet surface markers including rabbit anti-rat GPVI (Novus Bio, NBP1-76941), rabbit anti-CD42b (Santa Cruz, sc-292722) and rabbit anti-CD36 (Santa Cruz, sc-9154) followed by a 1-hour incubation with a goat anti-rabbit HRP conjugated secondary antibody. The Bio-Rad Mini-PROTEAN Tetra Cell system was used for a wet transfer. The SDS-PAGE gel was assembled into the apparatus with a PVDF membrane stacked between filter papers. For Immunocytochemistry staining, PNV-CSCs or CSCs were plated on 4-well culture 4-well slides (EMD Millipore, PEZGSO416). Slides were fixed with 4% PFA for 30 minutes at room temperature followed by permeabilization and blocking with Dako Protein block containing 0.1% saponin for 1 h at room temperature. A 4° C. overnight incubation of primary antibody using rabbit anti-rat GPVI (Novus Bio, NBP1-76941) and rabbit anti-CD42b (Santa Cruz, sc-292722) was followed by a 90 min incubation with a goat anti-rabbit Alexa fluora 488 conjugated secondary antibody (Abcam, ab150077). Nucleus is stained with DAPI for 10 minutes at room temperature (Life Technologies, R37606). Fluorescent images were taken by Olympus fluorescent microscope.
Collagen surface binding assay: GFP-tagged HUVECs (Angio-Proteomie, cAP-0001GFP, Boston, Mass.) were seeded on collagen-coated (Sigma Aldrich) 4-well culture chamber slide (Thermo Fisher Scientific) and cultured in Vascular Cell Basal Medium® (ATCC PCS-100-030) supplemented with endothelial cell growth kit-VEGF® (ATCC PCS-100-041). The cells were then incubated with DiI-loaded PNV-CSCs in PBS at 4° C. for 30 s. Next, the cells were washed with PBS twice and imaged using an Olympus Fluorescent Microscope. Attached PNV-CSCs were quantified.
Denuded rat aorta binding assay: To examine PNV-CSCs binding on injured (denuded) vascular walls, aortas from WKY rats were dissected and surgically scraped on their luminal side with forceps to remove the endothelial layer. Successful denudation was confirmed by microscopy visualization. Both denuded or control aortas were incubated with DiI-labeled PNV-CSCs or CSCs for 30 s. After PBS rinses, the samples were subjected to fluorescence microscopy examination for cell binding.
Rat model of ischemia/reperfusion: Acute myocardial infarction was induced by an ischemia/reperfusion procedure as previously described (Cheng et al., (2012) Cell Transplant 21: 1121-1135). Briefly, female WKY rats (6-8 weeks, Charles River Laboratories) underwent left thoracotomy in the 4th intercostal space under general anesthesia. The heart was exposed and myocardial infarction was produced by 60 min ligation of the left anterior descending coronary artery (LAD), using a 7-0 silk suture. The suture was then released to allow coronary reperfusion. Intracoronary injection was achieved by injection into the left ventricle cavity during a 25 s temporary aorta occlusion with a looped suture. Animals were randomized into three treatment groups: 1) Control, intracoronary injection of 200 μL PBS; 2) CSCs, intracoronary injection of 5×10⁵ CSCs in 200 μL PBS; 3) PNV-CSCs, intracoronary injection of 5×10⁵ PNV-CSCs in 200 μL PBS. The chest was closed and the animal was allowed to recover after all procedures. CSCs and PNV-CSCs were pre-labeled with CM-DiI. A cohort of animals were sacrificed 24 h after injection for ex vivo fluorescent imaging, qPCR and histological analysis of PNV-CSCs or CSCs retention while the rest of animals were followed for another 4 weeks.
Cell retention assay by fluorescence imaging (FLI) and quantitative PCR: Animals were sacrificed 24 h after cell infusion, the hearts were excised, washed with PBS, and placed in a Xenogen IVIS imaging system (Caliper Life Sciences, Mountain View, Calif.) to detect RFP fluorescence. Excitation was set at 550 nm and emission was set at 580 nm (Vandergriff et al., (2014) Biomaterials 35: 8528-8539). Exposure time was set at 5 s and kept the same during the entire imaging session.

Quantitative PCR was performed for precise measurement of the number of cells engrafted. As previously described (Vandergriff et al., (2014) Biomaterials 35: 8528-8539), CSCs derived from male donor WKY rats were injected into the myocardium of female recipients to utilize the detection of SRY gene located on the Y chromosome. The whole heart was weighed and homogenized. Genomic DNA was isolated from aliquots of the homogenate corresponding to 12.5 mg of myocardial tissue, using the DNA Easy Minikit (Qiagen), according to the manufacturer's protocol. The TaqMan® assay (Applied Biosystems, Carlsbad, Calif.) was used to quantify the number of transplanted cells with the rat SRY gene as template (forward primer, 5′-GGAGAGAGGCACAAGTTGGC-3′ (SEQ ID NO. 1), reverse primer: 5′-TCCCAGCTGCTTGCTGATC-3′ (SEQ ID NO. 2), TaqMan probe: 6FAM-CAACAGAATCCCAGCATGCAGAATTCAG-TAMRA (SEQ ID NO. 3); Applied Biosystems). For absolute quantification of cell number, a standard curve was constructed with samples derived from multiple dilutions of genomic DNA isolated from the male hearts. All samples were spiked with equal amounts of female genomic DNA to control for any effects this may have on reaction efficiency in the actual samples. The copy number of the SRY gene at each point of the standard curve was calculated with the amount of DNA in each sample and the mass of the rat genome per cell. For each PCR reaction, 50 ng of template DNA was used. Real-time PCR was performed with a real-time PCR System (Applied Biosystems). Cell numbers per mg of heart tissue and percentages of retained cells of the total injected cells were calculated.
Cardiac function assessment: The transthoracic echocardiography procedure was performed by a cardiologist who was blinded for animal group allocation using a Philips CX30 ultrasound system coupled with a L15 high-frequency probe. All animals underwent inhaled 1.5% isofluorane-oxygen mixture anesthesia in supine position at the 4 hours and 4 weeks. Hearts were imaged 2D in long-axis views at the level of the greatest left ventricular (LV) diameter. Ejection fraction (EF) was determined by measurement from views taken from the infarcted area.
Heart morphometry: After the echocardiography study at 4 weeks, animals were euthanized and hearts were harvested and frozen in OCT compound. Specimens were sectioned at 10 pm thickness from the apex to the ligation level with 100 μm intervals. Masson's trichrome staining was performed as described by the manufacturers instructions (HT15 Trichrome Staining (Masson) Kit; Sigma-Aldrich). Images were acquired with a PathScan Enabler IV slide scanner (Advanced Imaging Concepts, Princeton, N.J.). From the Masson's trichrome stained images, morphometric parameters including viable myocardium, scar size and infarct thickness were measured in each section with NIH ImageJ software. The percentage of viable myocardium as a fraction of the scar area (infarcted size) was quantified. Three selected sections were quantified for each animal.
Histology: For immunohistochemistry staining, heart cryosections were fixed with 4% paraformaldehyde, permeabilized and blocked with Protein Block Solution (DAKO, Carpinteria, Calif.) containing 0.1% saponin (Sigma), and then incubated with the mouse anti-alpha sarcomeric actin (a7811, Sigma), rabbit anti-Ki67 (ab15580, abcam) and rabbit anti-alpha smooth muscle Actin antibody (ab5694, abcam) overnight at 4° C. FITC or TxRed secondary antibodies were obtained from Abcam Company and used in conjunction with primary antibody. For assessment of angiogram, heart cryosections were incubated with Lectin (FL-1171, Vector laboratories, Burlingame, Calif., USA). Images were taken by an Olympus epi-fluorescence microscopy system.
CD42b blocking experiment: To explore which platelet adhesion molecules contributed to the targeting of PNV-CSCs, PNV-CSCs were pre-treated with anti-CD42b neutralizing antibodies (ab2578, mouse monoclonal [HIP1], Abcam) or isotype control antibodies (ab81032, mouse monoclonal, Abcam) for 30 min. After that, the cells were used in the ex vivo and in vivo experiments as previously described.
Porcine model of ischemia-reperfusion: Myocardial infarctions (MIs)were created in adult farm pigs (male, 3.5-4 months old) by inflation using an angioplasty balloon (TREK® OTW 3mm, Abbott Vascular, Santa Clara, Calif.) in the mid-left anterior descending artery (LAD) (distal to the second diagonal branch) for 1.5 hr. At the end of the ischemic period, the vessel was reperfused. The vessel was allowed for reperfusion for 15 mins prior to cell infusion and remain perfused throughout the study. Animals were randomized to two treatment groups: 10⁷ DiI-labeled CSCs or PNV-CSCs. The cells were delivered intracoronarily using an over-the-wire catheter (without balloon inflations, to exclude possible confounding effects related to ischemic post-conditioning). The cells were administered in 3 equally-divided cycles with wash solution infusion in between. The animals were euthanized and the hearts were excised and sliced for fluorescent imaging (for cell retention) and triphenyl tetrazolium chloride (TTC) staining (for infarct size measurement) 24 h after the procedure.
Statistical analysis: All results were expressed as mean ±standard deviation (s.d.). Comparison between two groups were conducted by two-tailed Student's t test. One-way ANOVA test was used for comparison among three or more groups with Bonferroni post hoc correction. Differences were considered statistically significant when P-values<0.05. The main efficacy endpoint was a change in LVEF as measured by echocardiography cardiac function assessment. Based on previous rodent echo studies, experimental groups differed by 10% on average in left ventricular ejection fractions with standard deviations as large as 5.0%. A sample size of 6 animals was calculated that would be needed in each experimental group (assuming a 5% confidence level and a 90% power level). Kolmogorov-Smirnov test was performed for normal distribution.
Animal randomization method: Animal cages were housed in a random order on the shelves. Physical randomization was performed before animal experiment using a paper-drawing method. All measurements were done in random order, with the surgeon and echocardiographer being blind to the treatment groups.

We claim:

1. A composition comprising:
(a) a platelet membrane-derived vesicle or a fragment thereof; and
(b) an animal or human cell, a plurality of said cells, or an extracellular vesicle derived from the cell,
wherein the platelet-derived membrane vesicle or a fragment thereof is fused into the outer membrane of the cell or plurality of said cells or encapsulates the extracellular vesicle, and wherein the composition is characterized as having specific binding affinity for at least one component of a vascular subendothelial matrix or vascular cell.

2. The composition of claim 1, wherein the extracellular vesicle is an exosome.

3. The composition of claim 1, wherein the animal or human cell is a stem cell.

4. The composition of claim 3, wherein the stem cell is a cardiac stem cell or a mesenchymal stem cell.

5. The composition of claim 1, wherein the animal or human cell has an outer membrane engineered to have platelet-derived polypeptide cell-surface markers.

6. The composition of claim 1, wherein the animal or human cell is isolated from an animal or human tissue, a cultured cell, or a cryopreserved cell.

7. The composition of claim 4, wherein the stem cell is from a cultured cardiosphere from a cardiac tissue.

8. The composition of claim 1, wherein the extracellular vesicle is derived from a cardiac stem cell or a mesenchymal stem cell.

9. The composition of claim 1, wherein the animal or human cell, plurality of cells, or the extracellular vesicle is derived from the same animal or human subject as the platelet-derived membrane vesicle.

10. The composition of claim 1, wherein the animal or human cell, plurality of cells, or the extracellular vesicle is derived from a different animal or human subject as the platelet-derived membrane vesicle or a fragment thereof.

11. The composition of claim 1, wherein (i) the animal or human cell, plurality of cells, or the extracellular vesicle and (ii) the platelet-derived membrane vesicle or a fragment thereof of the composition are both derived from the animal or human subject that is a recipient of the composition for treatment of a vascular injury.

12. The composition of claim 1, wherein at least one of (a) the animal or human cell, plurality of cells, or the extracellular vesicle and (b) the platelet-derived membrane vesicle or a fragment thereof of the composition is derived from the animal or human subject that is a recipient of the composition for treatment of a vascular injury.

13. The composition of claim 1, wherein the composition is admixed with a pharmaceutically acceptable carrier.

14. A method of generating a population of engineered animal or human cells or extracellular vesicles derived from said cells, the method comprising the step of mixing a population of platelet-derived membrane vesicles or fragments thereof and a population of cells or extracellular vesicles and thereby fusing the platelet-derived membrane vesicles or fragments thereof with the outer membranes of the cells or encapsulating the extracellular vesicles, wherein said cells or extracellular vesicles are isolated from an animal or human tissue or biofluid, cultured cells, or cryopreserved cells.

15. The method of claim 14, wherein the method further comprises the steps of:
(i) obtaining a suspension of platelets isolated from the plasma of an animal or human subject; and
(ii) sonicating the suspension of platelets to generate a population of platelet-derived membrane vesicles or fragments thereof.

16. The method of claim 14, further comprising the step of incubating the cells, or extracellular vesicles derived therefrom, together with the platelet-derived membrane vesicles or fragments thereof in the presence of polyethylene glycol (PEG) or extruding the cells, or extracellular vesicles derived therefrom, together with the platelet-derived membrane vesicles or fragments thereof, and thereby fusing the platelet-derived membrane vesicles or fragments thereof with the outer membranes of the cells or encapsulating the extracellular vesicles.

17. The method of claim 14, wherein the extracellular vesicle is an exosome.

18. The method of claim 14, wherein the animal or human cells are stem cells.

19. The method of claim 18, wherein the stem cells are derived from a cardiac tissue.

20. The method of claim 18, further comprising the step of obtaining the stem cells from a cultured tissue explant derived from a cardiac tissue.

21. The method of claim 14, comprising the step of obtaining the platelet-derived membrane vesicles and the cells, or the extracellular vesicles derived from said cells, from the same animal or human subject.

22. The method of claim 13, comprising the step of obtaining the platelet-derived membrane vesicles and the cells, or the extracellular vesicles derived from said cells, from different individual animal or human subjects.

23. A method of repairing a tissue injury in an animal or human subject, the method comprising administering to a recipient animal or human patient having a tissue injury a composition comprising a population of engineered cells or extracellular vesicles derived from said cells, wherein the engineered cells or extracellular vesicles comprise a platelet-derived membrane vesicle or a fragment thereof fused into the outer membrane of the cell or encapsulating the extracellular vesicle or population of said extracellular vesicles, and wherein the engineered cells or extracellular vesicles selectively target the subendothelial matrix or a vascular cell at the site of the tissue injury.

24. The method of claim 23, wherein the engineered cells are engineered stem cells or extracellular vesicles derived from said stem cells and the tissue injury of the subject is to a tissue of the cardiovascular system.

25. The method of claim 24, wherein the engineered cells are cardiac or mesenchymal stem cells and the extracellular vesicles derived from said cardiac or mesenchymal stem.

26. The method of claim 25, wherein the extracellular vesicles are exosomes.

27. The method of claim 23, wherein the tissue injury is accompanied by an injury to a blood vessel.

28. The method of claim 23, wherein the tissue injury is of neural tissue, muscular tissue, cardiac tissue, or hepatic tissue, and wherein the engineered cells migrate to the injured tissue.

29. The method of claim 23, wherein the engineered cells or engineered extracellular vesicles are derived from the same animal or human subject as the platelet-derived membrane vesicles.

30. The method of claim 23, wherein the engineered cells or engineered extracellular vesicles are not derived from the same animal or human subject as the platelet-derived membrane vesicles.

31. The method of claim 23, wherein at least one of (i) the engineered cells or engineered extracellular vesicles derived therefrom and (ii) the platelet-derived membrane vesicles or fragments thereof are derived from the recipient animal or human patient.


SEQUENCE LISTING (3 sequences)

SEQ ID NO: 1 - DNA, 20 bp, Artificial sequence
  Forward Taqman primer for rat SRY gene
ggagagaggcacaagttggc

SEQ ID NO: 2 - DNA, 19 bp, Artificial sequence
  Reverse Taqman Primer for rat SRY gene
tcccagctgcttgctgatc

SEQ ID NO: 3 - DNA, 28 bp, Artificial sequence
  Taqman probe for rat SRY gene with 6FAM and TAMIRA at 5′ and 3′ ends, respectively
caacagaatcccagcatgcagaattcag